(19) United States
(12) Patent Application Publication
(10) Pub. No.: US 2024/0141282 A1
(43) Pub. Date: May 2, 2024

(54) SPRAY FREEZE DRYING OF STRICT ANAEROBIC BACTERIA

(71) Applicant: Chr. Hansen A/S, Hoersholm (DK)

(72) Inventors: Michelle Milling Madsen, Hoersholm (DK); Kim Nielsen, Hoersholm (DK); Jimi Kjaersgaard Pettersson, Hoersholm (DK); Wendy Ossieur, Hoersholm (DK)

(73) Assignee: Chr. Hansen A/S, Hoersholm (DK)

(21) Appl. No.: 17/799,190

(22) PCT Filed: Feb. 25, 2021
(86) PCT No.: PCT/EP2021/054695

§ 371 Date: Aug. 11, 2022

(30) Foreign Application Priority Data
Feb. 26, 2020 (EP) 20159471.0

(51) Int. Cl. C12N 1/04; C12N 1/20

(52) U.S. Cl. CPC C12N 1/04; C12N 1/20

(57) ABSTRACT

The present invention provides a process for spray freezing or spray freeze drying under conditions of very low oxygen pressure, such as under essentially anaerobic conditions, and particularly a process for spray freezing strict anaerobic microorganisms (bacteria) under conditions where the level of oxygen is very low, or essentially anaerobic. Further, the invention provides a product achievable by the processes and an apparatus usable in the process.

### CROSS-REFERENCE TO RELATED APPLICATIONS

The present application is the U.S. National-Stage of International Application No. PCT/EP2021/054695, filed Feb. 25, 2021, and claims priority to European Patent Application No. 20159471.0, filed Feb. 26, 2020.

### FIELD OF THE INVENTION

The present invention provides a process for spray freezing or spray freeze drying under conditions comprising very low oxygen, such as under essentially anaerobic conditions, and particularly a process for spray freezing microorganisms sensitive to oxygen (e.g. strict anaerobic bacteria) under conditions where the level of oxygen is very low, or essentially anaerobic. Further, the invention provides a product achievable by the processes and an apparatus usable in the process.

### BACKGROUND OF THE INVENTION

Cryopreservation of lactic acid bacteria starter cultures has been used for many years to stabilize bacteria over longer time. A method that is commonly used is to form frozen pellets by allowing the lactic acid bacteria to drip into a container of liquid nitrogen. When the lactic acid bacteria are for use in applications where the larger pellets are not convenient, such as for example formulation of food supplements or pharmaceutical products, the resulting pellets are typically subsequently dried (e.g. by freeze drying) and then mechanically reduced in size, e.g. milled or grinded into smaller particles prior to formulation of the final product. This introduces a step in the production, which has the disadvantage of reducing the viability of the bacteria, e.g. due to shear forces applied.

Spray freezing is a technique wherein a suspension is atomized (or sprayed) and thereafter frozen, to produce frozen particles. Subsequently the frozen particles resulting from spray freezing can be stored in a freezer or dried (spray freeze drying), for example by lyophilization. An example of such a process is for example described in U.S. Pat. No. 7,007,406 (Wang), which discloses atmospheric spray freeze drying of liquid carrying pharmaceuticals to produce a powder of a pharmaceutical compound. Other examples of apparatus and studies where spray freeze drying has been used to dry food or bioproducts are disclosed by lshwarya et al. 2015.

Spray freeze drying has been proposed for freezing lactic acid bacteria, but with limited commercial success.

Volkert et al. 2008 discloses a study wherein Lactobacillus rhamnosus LGG® was spray frozen by atomizing a feed suspension in a sub-zero gas-atmosphere, where the spray equipment was placed in a cooling chamber to ensure temperatures of −30 to −35° C., and a gas pressure of 600 kPa was used. Frozen powder was gathered in a pick-up dish. Lactobacillus rhamnosus LGG® belongs to the group of lactic acid bacteria and is considered as an oxygen-tolerant bacteria and has strain-specific gene functions that are required to adapt to a large range of environments.

Semyonov et al. 2010 (Food Research International 43, 193-202 (2010)) have investigated the survival of Lactobacillus paracasei cells microencapsulated by spray freeze drying. Apparently, the bacterial suspension is sprayed by using a flow of air through a pneumatic nozzle into nitrogen in its liquid state. Lactobacillus paracasei belongs to the group of lactic acid bacteria and is considered as an oxygen-tolerant bacteria.

Her et al. 2015 discloses spray freeze drying of a suspension of Lactobacillus casei. Apparently, the bacterial suspension is sprayed by using a flow of air through a two-fluid nozzle and the resulting droplets were immersed in liquid nitrogen. L. casei belongs to the group of lactic acid bacteria and is considered as an oxygen-tolerant bacteria.

All the above spray freezing and drying processes have had limited commercial success, especially when the product to be preserved is bacteria cells which should be viable after thawing or rehydrating. Typically, during drying processes the lactic acid bacteria are susceptible to the oxidative, thermal, dehydration, shear and osmotic stress imposed during the drying, rethawing and rehydrating process.

WO2016083617 discloses a process for drying a microorganism containing suspension, characterized in that the aqueous suspension containing microorganisms is sprayed into a drying gas and subsequently into a cryogenic gas in a spray chamber. The frozen particles are collected and freeze dried until the water activity is below 0.2. WO2016083617 does not disclose a process for spray freeze drying a suspension comprising strict anaerobic bacteria.

Strict anaerobic bacteria (also called obligate anaerobe bacteria) are a group of bacteria that are highly sensitive to oxygen. Typically, the metabolic processes in these organisms have components that are extremely prone to oxidation or inactivation by molecular oxygen. Also, members of this group may lack important enzymes such as catalase involved in the inactivation of reactive oxygen species, such as superoxide anion, hydroxyl radical, and hydrogen peroxide. As a consequence, this group of bacteria is more difficult to ferment and preserve than lactic acid bacteria, especially on an industrial scale, since the exposure to oxygen from ambient air, and other types of oxidative conditions can be detrimental to the bacteria.

At the same time, many of the species belonging to the group of strict anaerobic bacteria have been suggested to have probiotic effects, for example by producing high amounts of butyrate and other anti-inflammatory compounds. There is thus a need for industrially scalable methods for stabilizing and formulating strict anaerobic bacteria e.g. for use in pharmaceutical or food supplement products.

Khan et al. 2014 relates to preservation of Faecalibacterium prausnitzii, and discloses a method where the bacteria are frozen at −20° C. and lyophilized for 3 h to form pellet-like granules or a foam-like matrix in an uneven size, and subsequently stored at −20° C.

As mentioned above, the disadvantage of freezing and drying bacteria in the form of a pellet or a foam-like matrix is that the resulting pellet of matrix will have to be mechanically reduced in size for use in a pharmaceutical product. In the case of strict anaerobic bacteria, this additional step would have to be performed under conditions preventing oxidative stress, such as for example in an anaerobic environment, or at least under the presence of very low levels of oxygen, which would further complicate the grinding step. These conditions further makes it a complex and costly step to include grinding in an industrial production of products comprising strict anaerobic bacteria.

There is thus still a need for improved production processes for strict anaerobic bacteria, or other microorganisms that are highly sensitive to oxygen.

### SUMMARY OF THE INVENTION

The present inventors have surprisingly discovered that strict anaerobic bacteria cells can be preserved effectively and resulting in a dry flowable powder of encapsulated bacteria with a surprisingly high vitality by a process which includes spray freezing in conditions of very low levels of oxygen.

Compared to prior art methods that involve pelletizing, freezing, drying and milling of the pellets, the process requires fewer steps and results in a powder that is free flowing and can be handled easier in industrial applications.

In one aspect, the invention relates to a process for preserving bacteria in a suspension comprising strict anaerobic bacteria, the process comprising the following steps:
a) forming droplets of a liquid suspension comprising said strict anaerobic bacteria by spraying or atomizing the suspension,
b) discharging the droplets into a chamber comprising cryogenic material to produce frozen particles; and
c) separating the frozen particles obtained in b) from the cryogenic material to obtain purified frozen particles.

This process simplifies the preservation process of the prior art by avoiding a step with a drying gas by simply discharging the droplets into cryogenic material to generate a suspension of frozen particles in cryogenic material.

In one aspect, the invention relates to a process for preserving bacteria in a suspension comprising strict anaerobic bacteria, the process comprising the following steps:
a) forming droplets of a liquid suspension comprising said strict anaerobic bacteria by spraying or atomizing the suspension
b) contacting the droplets with cryogenic material, such as cryogenic liquid and/or cryogenic gas, to produce frozen particles; and
c) separating the frozen particles obtained in b) from the cryogenic material to obtain purified frozen particles wherein the process steps a) to c) are performed in the presence of no more than about 2% oxygen, for example less than about 1% oxygen, preferably less than about 0.5% oxygen, e.g. less than about 0.05% oxygen. In other aspects, the invention relates to dry particles obtained by the process.

As shown in the examples, the processes, after drying of the frozen particles, results in a product with good particle properties and with acceptable viability even for strict anaerobic bacteria which are very sensitive to oxygen.

### Definitions

In general, the terms and phrases used herein have their art-recognized meaning, which can be found by reference to standard texts, journal references, and context known to those skilled in the art. The following definitions are provided to clarify their specific use in context of the disclosure.

As used herein, the singular forms “a”, “an” and “the” are intended to include the plural forms as well, unless the context clearly indicates otherwise.

The term “atomize” is in the present context to be construed as the act to convert a suspension or concentrate comprising microorganisms into very fine droplets, the droplets often comprising a microorganism (e.g. a bacteria, such as a strict anaerobic bacteria) and liquid.

The terms “comprising”, “having”, “including” and “containing” are to be construed as open-ended terms (i.e., meaning “including, but not limited to,”) unless otherwise noted.

“Extrusion” or “extruding” are terms well known in the art and refer to a process of forcing a composition, as described herein, through an orifice under pressure.

The terms “microorganism” or “microbe” in certain instances may refer to an organism of microscopic size, to a single-celled organism, and/or to any virus particle. The definition of microorganism used herein includes Bacteria, Archaea, single-celled Eukaryotes (protozoa, fungi, and ciliates), and viral agents. The term “microbial” in certain instances may refer to processes or compositions of microorganisms, thus a “microbial-based product” is a composition that includes microorganisms, cellular components of the microorganisms, and/or metabolites produced by the microorganisms.

The term “cryogenic material” as used herein refers to cryogenic liquids and cryogenic gases with a boiling point below −50° C. (−58° F.). The term is used interchangeably herein to refer to a single cryogenic liquid/cryogenic gas, or a plurality of cryogenic liquids and/or a plurality of cryogenic gases. Thus the term is not limiting to a specific number of or a specific type of cryogenic material. Typical cryogenic materials include helium, hydrogen, nitrogen, air, fluorine, argon, oxygen, methane, liquid natural gas, carbon dioxide, nitrous oxide and nitrous carbon. A common property of cryogenic materials is that they are in gaseous phase at normal room temperatures and pressures, and that they have a boiling point below −50° C., at 1 atm pressure. However, most cryogenic materials have a boiling point below −150° C. (−238° F.), at 1 atm pressure.

The term “cryogenic liquid” as used herein refers to liquefied gas that is kept in its liquid state. Cryogenic liquids has a boiling point below −50° C. (−58° F.), and typically below −150° C. (−238° F.).

The term “cryogenic gas” as used herein refers to a cryogenic material in gaseous phase, e.g. a cryogenic liquid that has vaporized.

In the present context, the term “packaging” (a suitable amount of) the dried microorganism in a suitable package relates to the final packaging to obtain a product that can be shipped to a customer. A suitable package may thus be a container, bottle or similar, and a suitable amount may be e.g. 0.1 g to 30000 g. The term package includes a bag, a box, a capsule, a pouch, a sachet, a container, etc.

“Pellet”: The terms “pellet” and/or “pelleting” refer to solid rounded, spherical and/or cylindrical tablets or pellets and the processes for forming such solid shapes, particularly larger particles.

As used herein, the term “product” in certain instances may refer to a microbial composition that can be blended with other components and contains specified concentration of viable cells that can be sold and used.

“Strict anaerobic bacteria” (also called obligate anaerobe bacteria) is a group of bacteria that are sensitive to oxygen, particularly strict anaerobic bacteria genera that do not express catalase.

A “stable” formulation or composition is one in which the biologically active material therein essentially retains its physical stability, chemical stability, and/or biological activity upon storage.

Stability can be measured at a selected temperature and humidity conditions for a selected time period. Trend analysis can be used to estimate an expected shelf life before a material has actually been in storage for that time period. For live bacteria, for example, stability may be defined as the time it takes to lose 1 log of CFU/g dry formulation under predefined conditions of temperature, humidity and time period.

“Viability” with regard to bacteria, refers to the ability to form a colony (CFU or Colony Forming Unit) on a nutrient medium appropriate for the growth of the bacteria. Alternatively, viability may be measured as the most probable number (MPN) or using flow cytometry. Viability, with regard to viruses, refers to the ability to infect and reproduce in a suitable host cell, resulting in the formation of a plaque on a lawn of host cells.

The term “viable cell” may in certain instances mean a microorganism that is alive and capable of regeneration and/or propagation, while in a vegetative, frozen, preserved, or reconstituted state.

The term “viable cell yield” or “viable cell concentration” may, in certain instances refer to the number of viable cells in a liquid culture, concentrated, or preserved state per a unit of measure, such as liter, milliliter, kilogram, gram or milligram. The term “cell preservation” in certain instances may refer to a process that takes a vegetative cell and preserves it in a metabolically inert state that retains viability over time.

### DETAILED DESCRIPTION OF THE INVENTION

The present invention relates to stabilization of microorganisms that are sensitive to oxygen, such as for example a species of a facultative anaerobic bacteria or more preferably a species of strict anaerobic bacteria. Such a process is useful for the stabilizing and preserving bacteria with high viability in a powder format that can e.g. be dosed and formulated for pharmaceutical purposes.

In certain examples of aspects of the invention, the microorganism is a strict anaerobic bacteria. Strict anaerobic bacteria (also called obligate anaerobe bacteria) is a group of bacteria that are sensitive to oxygen. Typically, the metabolic processes in these organisms have components that are extremely sensitive to oxidation or inactivation by molecular oxygen. Also, members of this group may lack important enzymes e.g. catalase involved in the inactivation of reactive oxygen species, such as superoxide anion, hydroxyl radical, and hydrogen peroxide. In some examples of the invention, the microorganism is as a species of strict anaerobic bacteria.

Accordingly, the microorganism is at least one microorganism selected from the group of strict anaerobic bacteria consisting of Adlercreutzia sp., Akkermansia sp., Alistipes sp., Anaerotruncus sp., Bacteroidales, Bacteroides sp., Blautia sp., Butyricicoccus sp., Butyrivibrio sp., Catabacteriaceae sp., Christensenella sp., Clostridiales sp., Clostridium sp., Collinsella sp., Coprococcus sp., Cutibacterium sp., Dialister sp., Dorea sp., Erysipelotrichaceae sp. Eubacterium sp., Faecalibacterium sp., Flavonifractor sp., Fusobacterium sp., Hafnia sp., Holdemania sp., Hungatella sp., Intestinibacter sp., Lachnobacterium sp., Lachnospira sp., Lachnospiraceae sp, Lachnospiraceae gen. nov. sp. Nov, Lachnospiraceae sp. nov., Methanobrevibacter sp., Methanomassiliicoccus sp., Methanosarcina sp., Mitsuokella sp., Odoribacter sp., Oscillospira sp., Oxalobacter sp., Parabacteroides sp., Phascolarctobacterium sp., Porphyromonadaceae sp., Prevotella sp., Propionibacterium sp., Rikenellaceae sp., Roseburia sp. Ruminococcus sp., Subdoligranulum sp., Sutterella sp., and Turicibacteraceae sp.

In another example the microorganism is at least one microorganism selected from the group of strict anaerobic bacteria consisting of Adlercreutzia sp., Adlercreutzia equolifaciens, Akkermansia sp., Akkermansia muciniphila, Alistipes sp., Alistipes finegoldii, Alistipes hadrus, Alistipes indistinctus, Alistipes onkerdonkii, Alistipes putredinis Alistipes shahii, Anaerostipes sp. Anaerostipes caccae, Anaerostipes hadrus, Anaerotruncus sp., Bacteroidales, Bacteroides sp., Bacteroides dorei, Bacteroides fragilis, Bacteroides intestinalis, Bacteroides intestinihominis, Bacteroides ovatus, Bacteroides putredinis, Bacteroides thetaiotaomicron, Bacteroides uniformis, Bacteroides vulgatus, Bacteroides xylanisolvens, Blautia sp, Blautia luti, Blautia obeum, Blautia wexlerae, Butyricicoccus, Butyrivibrio fibrisolvens, Butyrivibrio sp., Catabacteriaceae, Christensenella sp., Clostridiales, Clostridium sp., Clostridium scindens, Clostridium spiroforme, Clostridium butyricum, Collinsella sp., Collinsella aerofaciens, Coprococcus sp., Coprococcus catus, Coprococcus comes, Coprococcus eutactus, Coprococcus sp., Cutibacterium acnes, Dialister sp., Dialister invisus, Dorea sp., Dorea formicigenerans, Dorea longicatena, Erysipelotrichaceae, Eubacterium sp. Eubacterium eligens, Eubacterium hallii, Eubacterium limosum, Eubacterium ramulus, Eubacterium rectale, Eubacterium siraeum, Eubacterium ventriosum, Faecalibacterium sp., Faecalibacterium prausnitzii, Flavonifractor plautii, Fusobacterium prausnitzii, Hafnia, Holdemania, Hungatella hathewayi, Intestinibacter bartlettii, Lachnobacterium, Lachnospira, Lachnospira pectinoshiza, Lachnospiraceae, Lachnospiraceae gen. nov. sp. Nov, Lachnospiraceae sp. nov., Methanobrevibacter sp., Methanomassiliicoccus sp., Methanosarcina, Mitsuokella multiacidus, Odoribacter, Oscillospira, Oxalobacter formigenes, Parabacteroides sp., Parabacteroides distasonis, Phascolarctobacterium, Porphyromonadaceae, Prevotella sp., Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella veroralis, Propionibacterium acnes, Rikenellaceae, Roseburia sp. Roseburia faecis, Roseburia hominis, Roseburia intestinalis, Roseburia inulinivorans, Ruminococcus sp., Ruminococcus bicirculans, Ruminococcus gauvreauii, Ruminococcus gnavus, Ruminococcus lactaris, Ruminococcus obeum, Ruminococcus torques, Ruminococcus albus, Ruminococcus bromii, Ruminococcus callidus, Ruminococcus flavefaciens, Ruminococcus gauvreauii, Subdoligranulum, Sutterella and Turicibacteraceae.

In other examples of the invention, the microorganism is at least one microorganism selected from the group of strict anaerobic bacteria consisting of Faecalibacterium prausnitzii, Eubacterium hallii.

In certain examples of the invention a microorganism is not a member of the genus Bifidobacterium. In other certain examples of the invention a microorganism is not a species of Bifidobacterium animalis.

Process for Stabilizing an Oxygen Sensitive Microorganism

In one aspect, the invention relates to a process for preserving bacteria in a suspension comprising strict anaerobic bacteria, the process comprising the following steps:
a) forming droplets of a liquid suspension comprising said strict anaerobic bacteria by spraying or atomizing the suspension,
b) discharging the droplets into a chamber comprising cryogenic material to produce frozen particles; and
c) separating the frozen particles obtained in b) from the cryogenic material to obtain purified frozen particles.

This process simplifies the preservation process of the prior art by avoiding a step with a drying gas.

In one aspect, the invention relates to a process for preserving bacteria in a suspension comprising strict anaerobic bacteria, the process comprising the following steps:
a) forming droplets of a liquid suspension comprising said strict anaerobic bacteria by spraying or atomizing the suspension
b) contacting the droplets with cryogenic material, such as cryogenic liquid and/or a cryogenic gas to produce frozen particles; and
c) separating the frozen particles obtained in b) from the cryogenic material to obtain purified frozen particles
wherein the process steps a) to c) are performed in the presence of no more than about 2% oxygen, for example less than about 1% oxygen, preferably less than about 0.5% oxygen, e.g. less than about 0.05% oxygen. In other aspects, the invention relates to dry particles obtained by the process.

In step b) of the process, the frozen particles are formed by contacting the droplets with cryogenic material, such as liquid nitrogen and/or a cryogenic gas. The droplets can be sprayed into the gas phase of a cryogenic gas or into liquid nitrogen.

In further examples of the present disclosure, the cryogenic material is selected from the group of helium, hydrogen, nitrogen, air, fluorine, argon, oxygen, methane, carbon dioxide, nitrous oxide and/or nitrous carbon. The cryogenic material may be in gaseous phase and/or liquid phase, thus the cryogenic material may be one or more cryogenic liquids and/or one or more cryogenic gases. The cryogenic material has a boiling point below −50° C. (−58° F.), and typically below −150° C. (−238° F.), at 1 atm pressure. In a specific example of the present disclosure, the cryogenic material is liquid nitrogen.

In some examples of the invention, process step b) is performed by contacting the droplets with cryogenic material, such as cryogenic liquid and/or a cryogenic gas, to produce frozen particles. It is a preference that the cryogenic material, such as liquid and/or the cryogenic gas, has a temperature of −50° C. (−58° F.) or lower, more preferably −75° C. (−103° F.) or lower, yet more preferably −100° C. (−148° F.) or lower, even yet more preferably −125° C. (−193° F.) or lower, most preferably −150° C. (−238° F.) or lower. In some examples of the invention, at least the process steps a) to c), or at least the process steps a) to d), or all process steps a) to e) are performed in the presence of less than about 0.5% oxygen, such as less than about 0.25% oxygen, such as less than about 0.1% oxygen, such as less than about 0.05% (about 500 ppm) oxygen, such as less than about 0.02% oxygen, or less than about 0.03% oxygen, or less than about 0.04% oxygen.

In other preferred examples of the invention, at least the process steps a) to c), or at least the process steps a) to d), or all process steps a) to e) are performed in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%.

In still other preferred examples of the invention, at least the process steps a) to c), or at least the process steps a) to d), or all process steps a) to e) are performed in the presence of oxygen in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%.

In still other preferred examples of the invention, at least the process steps a) to c), or at least the process steps a) to d), or all process steps a) to e) are performed under essentially anaerobic conditions.

In still other preferred examples of the invention, at least the process steps a) to c), or at least the process steps a) to d), or all process steps a) to e) are performed in an environment essentially free of oxygen, such as in the presence of a gas that is not oxygen, such as for example nitrogen gas.

The pressure surrounding the atomized suspension influences physical properties of the suspension, e.g. the evaporation or sublimation temperature of water in droplets formed in step a).

In some examples of the invention process step a) is performed in a chamber having a pressure kept in the range between about 60 kPa to about 400 kPa, for example in the range between 60 kPa to 200 kPa, such as in the range between 80 kPa and 120 kPa, preferably in the range between 90 kPa to about 110 kPa, e.g. at about 101 kPa (atmospheric pressure).

In some examples of the invention process step b) is performed in a chamber having a pressure kept in the range between about 60 kPa to about 400 kPa, for example in the range between about 60 kPa to 200 kPa, such as in the range between 80 kPa and 120 kPa, preferably in the range between 90 kPa to about 110 kPa, e.g. at about 101 kPa (atmospheric pressure).

In preferred examples of the invention, steps a), b) and c) are performed in a chamber having a pressure kept in the range between about 60 kPa to about 400 kPa, for example in the range between about 60 kPa to 200 kPa, such as in the range between 80 kPa and 120 kPa, preferably in the range between 90 kPa to about 110 kPa, e.g. at about 101 kPa (atmospheric pressure). The chamber may contain cryogenic material, such as a cryogenic gas, while being contacted from the outside by cryogenic material, such as cryogenic liquid. For example the chamber may be immersed in cryogenic material, such as cryogenic liquid. The cryogenic material outside the chamber may thereby cool the interior of the chamber including the contained cryogenic material. Preferably the cryogenic material outside the chamber has a temperature of −50° C. (−58° F.) or lower, more preferably −75° C. (−103° F.) or lower, yet more preferably −100° C. (−148° F.) or lower, even yet more preferably −125° C. (−193° F.) or lower, most preferably −150° C. (−238° F.) or lower. Similarly, it is a preference that the cryogenic material contained by the chamber has a temperature of −50° C. (−58° F.) or lower, more preferably −75° C. (−103° F.) or lower, yet more preferably −100° C. (−148° F.) or lower, even yet more preferably −125° C. (−193° F.) or lower, most preferably −150° C. (−238° F.) or lower.

In some examples of the invention, steps a) and b) are performed in the same chamber, e.g. by spraying the suspension into a chamber containing cryogenic material, e.g. liquid nitrogen and/or a cryogenic gas, e.g. nitrogen gas. In such examples of the invention, the chamber may have a pressure kept in the range between 90 kPa to about 110 kPa, e.g. at about 101 kPa (atmospheric pressure).

In other examples of the invention, the droplets are sprayed directly into cryogenic material, such as cryogenic liquid and/or cryogenic gas, which said cryogenic material is not necessarily housed in a closed chamber.

The process of the present invention involves formation of droplets of the suspension comprising a bacteria. In preferred examples of the invention, the droplets are formed by spraying the liquid suspension. In such examples of the invention, the formation or preparation of droplets is carried out by means of a spray nozzle (atomizing device), such as an ultrasound nozzle; a pressure nozzle; a two-fluid nozzle (e.g. using N₂ as atomizing gas); a vibrating nozzle; a frequency nozzle, an electrostatic nozzle; or a rotating atomizing device.

In some embodiments of the invention, the formation or preparation of droplets is carried out by means of a two-fluid nozzle typically functioning to atomize a liquid, e.g. a suspension of bacteria, by causing the interaction of high velocity gas and liquid.

Atomizing the suspension comprising a bacteria (such as a strict anaerobic bacteria) results in the formation or preparation of droplets having a size from about 5 to about 500 micrometer, such as in the range from about 5 to about 400 micrometer, such as about 10 to about 350 micrometer, about 10 micrometer to about 300 micrometer, about 10 micrometer to about 200 micrometer, such as about 10 micrometer to about 50 micrometer, or such as about 50 micrometer to about 200 micrometer, such as about 50 micrometer to about 100 micrometer, such as about 75 micrometer, or such as about 100 micrometer to about 200 micrometer, such as about 150 micrometer, measured as Dv50 values in micrometer.

In a specific example of the invention, the formation of droplets is carried out by means of a spray nozzle (atomizing device), and the prepared droplets has a size of between 5 and 800 micrometers, for example 5 and 600 micrometers, such as 5 and 400 micrometers and preferably between 10 and 250 micrometer, measured as Dv50 values in micrometer.

In certain examples of the invention, the formation of droplets in step a) is performed using a spray gas (atomizing gas), e.g. in combination with a two-fluid nozzle. Such a spray gas can be selected from the group consisting of an inert gas (such as nitrogen), a noble gas (e.g. helium, argon or neon), carbon dioxide, and an alkane gas (such methane), a cryogenic gas and a mixture thereof.

In certain examples of the invention, the spray gas comprises or consists of nitrogen gas.

As the spray gas comes into contact with the suspension of bacteria during the formation of the droplets, the inlet temperature of the spray gas may influence the rate of drying that occurs in the droplets prior to freezing.

In certain examples of the invention, the droplet forming step a), (e.g. the spray step) is carried out at a spray gas inlet temperature of at most about 80° C., such as in the range between about 0° C. to about 60° C., such as in the range between about 0° C. to about 15° C., or such as in the range between about 15° C. to about 30° C., such as between about 18° C. to about 25° C., such as about 19° C., about 20° C., about 21° C., about 22° C., about 23° C., or about 24° C., such as at about 22° C. (room temperature).

In certain examples of the invention, the droplet forming step a), (e.g. the spray step) is carried out at with a spray gas inlet temperature in the range between about 15° C. to about 30° C., preferably such as in the range between about 18° C. to about 25° C., such as at about 22° C.

The inlet pressure of the spray gas influences the rate of flow through the nozzle, and may influence the size of the droplets formed, as well as the stress on the bacteria during the formation of droplets.

In certain examples of the invention, the spray gas has an inlet pressure in the range between about 1 kPa to about 500 kPa, such as in the range between about 5 kPa to about 500 kPa, such as in the range between about 5 kPa to about 300 kPa, such as in the range between about 5 kPa to about 100 kPa, such as about 60 kPa, or such as about 70 kPa, or such as about 80 kPa, or such as in the range between about 100 kPa to about 400 kPa, such as about 120 kPa, or about 150 kPa, or about 200 kPa, or about 250 kPa, or about 300 kPa, or about 350 kPa.

In certain examples of the invention, spray gas has an inlet pressure in the range between about 100 kPa to about 400 kPa.

The freezing of the droplets formed by the liquid suspension results in frozen particles of a certain water content. In some examples of the process, the water content of the suspension prior to freezing is between about 5% and about 98%, for example between about 10% and about 95% by weight, preferably between about 30% and about 80%, or between about 40% and about 75% percent by weight, with respect to the total weight of the frozen particle(s).

Microorganisms are often preserved with an addition of additive compounds that in various ways may help to stabilize the microorganisms during the processes of freezing, drying, thawing and rehydration to increase the viability of the microorganisms. These additives may for example be referred to as cryoprotectant, drying protectant or cryoformulation.

Thus, in certain examples of the invention, the suspension comprising microorganisms such as strict anaerobic bacteria further comprises one or more stabilizing additives. Thus, in certain examples of the invention, one or more additives are added to the bacterial suspension prior to formation of droplets of step a).

In some examples of the invention, one or more additives are added to the suspension prior to step a) in the presence of less than about 5% oxygen, such as less than about 2% oxygen, preferably less than about 1% oxygen, such as 0.5% oxygen, such as less than about 0.05% oxygen.

In other examples of the invention, one or more additives are added to the suspension prior to formation of droplets of step a) in the presence of less than about 0.5% oxygen, such as less than about 0.25% oxygen, such as less than about 0.1% oxygen, such as less than about 0.05% (about 5 ppm) oxygen, such as less than about 0.02% oxygen, or less than about 0.03% oxygen, or less than about 0.04% oxygen.

In still other examples of the invention, one or more additives are added to the suspension prior to step a) in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01% oxygen, or such as about 0.02% oxygen, or in the range between about 0.025% to about 0.05% oxygen, such as about 0.03% oxygen, or such as about 0.04% oxygen. In certain other examples of the invention, one or more additives are added to the suspension prior to formation of droplets in step a), under essentially anaerobic conditions.

In certain examples of the invention, various additives may be added or mixed with the suspension comprising microorganisms, e.g. strict anaerobic bacteria. Thus, in some examples of the invention, such one or more additives is selected from the group consisting of inositol, lactose, sucrose, trehalose, inulin, maltodextrin, dextrose, alginate or a salt thereof (e.g. sodium alginate), skimmed milk powder, yeast extract, casein peptone, hydrolyzed protein, such as hydrolyzed casein, casein or salts thereof (such as sodium caseinate), inosine, inosinemonophospate and a salt thereof, glutamine and salts thereof (such as monosodium glutamate), ascorbic acid and salts thereof (such as sodium ascorbate), citric acid and salts thereof, propyl gallate or salts thereof, polysorbate, a hydrate of Magnesium sulphate (e.g. a heptahydrate), a hydrate of manganous sulphate (e.g. a monohydrate) and dipotassium hydrogen phosphate, propyl gallate and combinations thereof.

In some examples of the invention, the one or more additives is selected from the group consisting of yeast extract, dextrose, polysorbate, dipotassium hydrogen phosphate, magnesium sulphate heptahydrate, manganous sulphate monohydrate, and combinations thereof.

In some examples of the invention, the one or more additives is selected from the group consisting of yeast extract, dextrose, polysorbate, dipotassium hydrogen phosphate, magnesium sulphate heptahydrate, manganous sulphate monohydrate and optionally a mixture of vitamins.

Step c)

In the step c) of the process, the frozen particles obtained in step b) are separated or isolated from the cryogenic material, such as liquid nitrogen, to obtain purified frozen particles. In some examples of the process, the frozen particles are separated from the cryogenic material, such as liquid nitrogen, using a filter (such as an electrostatic filter) or sieve.

In certain examples of the process, the frozen particles obtained in b) are separated from the cryogenic material, such as liquid nitrogen, and collected using a sieve, such as a sieve having an aperture diameter below about 500 micrometer, such as in the range between about 10 and about 400 micrometer, such as in the range between about 40 micrometer to about 300 micrometer, such as in the range from about 50 micrometer to about 250 micrometer, such as about 50 micrometer, such as about 100 micrometer, such as about 150 micrometer, such as about 200 micrometer or such as about 250 micrometer.

In more specific examples of the process, the frozen particles obtained in step b) are separated from the cryogenic material, such as liquid nitrogen, using a sieve, such as a sieve having an aperture diameter in the range from about 40 micrometer to about 300 micrometer.

The freezing of the droplets formed by the liquid suspension results in frozen particles of a certain water content. In some examples of the process, the water content of the purified frozen particle(s) is between about 5% and about 98% by weight, such as 10% and about 95% by weight, preferably between about 30% and about 80%, or between about 40% and about 75% percent by weight, with respect to the total weight of the purified frozen particle(s).

Step d)

The frozen particles of the present invention may optionally further be dried using various techniques, e.g. such as freeze drying or fluidized bed drying, to produce dried particles.

In certain examples of the invention, the process comprises a drying step to produce dried particles. During such a drying step, the water content of the particles is typically reduced by evaporating or sublimation of water. Preferably, drying of the purified frozen particles to produce dried particles is performed under reduced pressure, such as by freeze-drying (lyophilization).

The drying step is performed to reduce the water content and/or water activity of the product, which is decreased to improve the stabilization of a microorganism, e.g. a strict anaerobic bacteria. In some examples of the process, the drying of the purified frozen particles is performed until the water activity (aw) is below about 0.8, such as below 0.6, such as in the range of about 0.01 to about 0.8, such as about 0.05 to about 0.5, such as about 0.1, or such as about 0.2, or such as about 0.3, or such as about 0.4.

In some examples of the process, the drying of the purified frozen particles is performed until the e.g. water content of the dried particles is between about 0.1% and about 30% by weight, such as in the range between about 1% to about 15% by weight, such as in the range between about 5% to about 10% by weight, or such as in the range between about 0.1% to about 5% by weight, with respect to the total weight of the particles.

As a consequence, in an embodiment of the present disclosure the dried particles include a microorganism having a viability of at least 1.0×10E4 per gram, such as defined by the most probable number (MPN), the number of colony forming units (CFU) or the number of viable cells measured by standard lab tools such as flow cytometry.

In an embodiment of the present disclosure the dried particles include a microorganism having a viability above 1.0×10E4 per gram, such as defined by the most probable number (MPN), the number of colony forming units (CFU) or the number of viable cells measured by standard lab tools such as flow cytometry, such as in the range between 1.0×10E4 to 1.0×10E13, such as in the range between about 1.0×10E4 to about 1.0×10E10 per gram, such as about 1.0×10E5, about 1×10E6, about 1.0×10E7, about 1×10E8, about 1.0×10E9, about 2.5×10E9, about 5.0×10E9, or about 7.5×10E9 per gram.

In an embodiment of the present disclosure the dried particles include a microorganism having a viability in the range between about 1.0×10E4 and about 1.0×10E13, such as about 10E6 to about 10E10, e.g. about 10E7 per gram, such as defined by the most probable number (MPN), the number of colony forming units (CFU) or the number of viable cells measured by standard lab tools such as flow cytometry.

The suspensions comprising microorganisms (e.g. strict anaerobic bacteria) may be concentrated prior to the formation of droplets. Such a concentration has the function to remove water and components of the culture medium which has been used for culturing the microorganisms. Thus, in some examples of the invention, the process further comprises a concentrating step prior to the formation of droplets (atomization) in step a), wherein a suspension of microorganisms is concentrated by removing fluid from the suspension, e.g. by centrifugation or filtration.

In some examples of the invention, the process further comprises a concentrating step prior to droplet formation (e.g. atomization) step a), wherein a suspension of microorganisms (e.g. strict anaerobic bacteria) is concentrated by removing fluid from the suspension, e.g. by centrifugation or filtration in the presence of less than about 5% oxygen, such as less than about 2% oxygen, preferably less than about 1% oxygen, such as 0.5% oxygen, such as less than about 0.05% oxygen.

In some more specific examples of the invention, the process further comprises a concentrating step prior to droplet formation (e.g. atomization) step a), wherein a suspension of microorganisms or protein is concentrated in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%.

In some more specific examples of the invention, the process further comprises a concentrating step prior to droplet formation (e.g. atomization) step a), wherein the concentration step is performed in essentially anaerobic conditions.

A further washing step may also be included prior to the droplet formation step a) washing step prior to the formation of droplets (atomization) in step a), wherein a suspension of microorganisms (e.g. strict anaerobic bacteria), such as a concentrated suspension of microorganisms is washed to remove components from the suspension of microorganism, e.g. components of the culture medium.

In some examples of the invention, the process further comprising a washing step prior to droplet formation (e.g. atomization) step a), wherein a suspension of microorganisms (e.g. strict anaerobic bacteria), for example a concentrated suspension of microorganisms, is washed to remove components of the culture medium, while maintaining the microorganisms (e.g. strict anaerobic bacteria) in the suspension, in the presence of less than about 5% oxygen, such as less than about 2% oxygen, preferably less than about 1% oxygen, such as 0.5% oxygen, such as less than about 0.05% oxygen.

In some more specific examples of the invention, the process further comprises a washing step prior to droplet formation (e.g. atomization) step a), wherein a suspension of microorganisms (e.g. strict anaerobic bacteria), such as a concentrated suspension of microorganisms is washed in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%.

In some more specific examples of the invention, the process further comprises a washing step prior to droplet formation (e.g. atomization) step a), wherein the concentration step is performed in essentially anaerobic conditions.

In order to propagate microorganisms sensitive to oxygen (e.g. strict anaerobic bacteria) and provide viable material for the production process of the invention, the microorganisms sensitive to oxygen (e.g. strict anaerobic bacteria) are fermented in a culture medium prior to e.g. concentration and/or stabilization.

For microorganisms sensitive to oxygen (e.g. strict anaerobic bacteria) such a fermentation step may be performed in a manner that protects the microorganisms from oxygen. Thus, in some examples of the invention, the process further comprises a fermentation step prior to step a), wherein the suspension is fermented in the presence of less than about 5% oxygen, such as less than about 2% oxygen, preferably less than about 1% oxygen, such as less than about 0.5% oxygen, such as less than about 0.05% oxygen.

In some examples of the invention, the process further comprises a fermentation step prior to step a), wherein the suspension is fermented under essentially anaerobic conditions.

In order to increase the viability and decrease stress of the oxygen sensitive microorganism (e.g. strict anaerobic bacteria), the process of the invention may include a fermentation step, concentration step, and the process steps a) to b), or process steps a) to c), or process steps a) to d), or process steps a) to e), which are all performed in the presence of no more than 0.5% oxygen, such as less than 0.05% oxygen.

In a more specific example of the invention, the process of the invention may include a fermentation step, concentration step, and the process steps a) to b), or process steps a) to c), or process steps a) to d), or process steps a) to e) which are all performed are performed under essentially anaerobic conditions.

When a washing step is further included in the process, the process of the invention may include a fermentation step, concentration step, a washing step, and the process steps a) to b), or process steps a) to c), or process steps a) to d), or process steps a) to e), which are all performed in the presence of no more than 0.5% oxygen, such as less than 0.05% oxygen.

When a washing step is further included in the process, the process of the invention may include a fermentation step, concentration step, a washing step, and the process steps a) to b), or process steps a) to c), or process steps a) to d), or process steps a) to e), which are all performed under essentially anaerobic conditions.

### Product Obtainable by a Process of the Invention

A further aspect of the present invention is the particle product obtainable by the process of the invention as described herein. Such particles comprise a bacterium sensitive to oxygen, such as a strict anaerobic bacteria and may be frozen or dried, isolated or comprised in the cryogenic material, such as liquid nitrogen. In certain examples of the invention, the particle is a frozen particle or a dried particle, and more specifically the particle is a dry particle.

The particle of the present invention, may comprise a single species of a microorganism (e.g. a single species of strict anaerobic bacteria), or a plurality of species of microorganisms (e.g. a plurality of species of strict anaerobic bacteria).

Particles according to the invention may comprise at least one species of a strict anaerobic bacteria. In certain examples of the invention, the particles (e.g. the dried particles) consist of at least one microorganism (e.g. a strict anaerobic bacteria) and one or more additives.

The particle according to the invention may have a size from about 5 to about 800 microns, such as about 5 to about 600 microns, such as 5 to about 500 micrometer, such as in the range from about 5 to about 400 micrometer, such as about 10 to about 350 micrometer, about 10 micrometer to about 300 micrometer, about 10 micrometer to about 250 micrometer, such as about 10 micrometer to about 50 micrometer, or such as about 50 micrometer to about 200 micrometer, such as about 50 micrometer to about 100 micrometer, such as about 75 micrometer, or such as about 100 micrometer to about 200 micrometer, such as about 150 micrometer, measured as Dv50 values in micrometers.

In certain examples of the invention, the particle has a size of between about 5 to about 400 micrometers, preferably between about 10 to about 250 micrometer, as measured as Dv50 values in micrometer.

The liquid (e.g. water) content of the dried particles influence the stability of the bacteria (e.g. strict anaerobic bacteria). Accordingly, the dried particles according to the invention may have a liquid (e.g. water) content between about 0.1% and about 30% by weight, such as in the range between about 1% to about 15% by weight, such as in the range between about 5% to about 10% by weight, or such as in the range between about 0.1% to about 5% by weight, with respect to the total weight of the particle.

Thus, in one example of the invention, the dried particle comprise at least one species of strict anaerobic bacteria, and may have a size of between about 5 to about 400 micrometer, preferably between about 10 micrometer to about 200 micrometer, as measured as Dv50 values in micrometer, and further having a liquid (e.g. water) content between about 0.1% and about 30% by weight, such as in the range between about 1% to about 15% by weight, such as in the range between about 5% to about 10% by weight, or such as in the range between about 0.1% to about 5% by weight, with respect to the total weight of the particle.

Powder properties, such as the flow properties, density, cohesive strength and wall friction of a powder may influence the handling and processing of the dried particles comprising a microorganism, such as a strict anaerobic bacteria of the present invention. The flow properties of the particles arise from the collective forces acting on individual particles (e.g. van der Waals, electrostatic, surface tension, interlocking and friction.)

In certain examples of the invention, the dry particles have reduced aggregation and a relatively narrow size distribution.

In some examples of the invention, a plurality of particles of the invention form a free-flowing powder.

### An Apparatus for Use in a Process of the Invention

A further aspect of the invention provides an apparatus usable in the process of the invention. Such an apparatus may comprise a chamber, the chamber comprising i) an atomizing means for spraying or atomizing the suspension, ii) optionally an inlet for a spray gas, iii) an inlet for cryogenic material, i.e. cryogenic liquid and/or cryogenic gas, and iv) an outlet for the frozen particles.

The processes and apparatus of the invention are specially designed for oxygen sensitive bacteria, such as a strict anaerobic bacteria. Accordingly, the means for performing steps a) to b), or a) to c), or a) to d) are suited for reducing the amount of oxygen in contact with the microorganism (e.g. a strict anaerobic bacteria). Thus, in certain examples of the invention, steps a) to b), or a) to c), or a) to d) are performed in the presence of less than about 0.5% oxygen, such as less than about 0.25% oxygen, such as less than about 0.1% oxygen, such as less than about 0.05% (about 500 ppm) oxygen, such as less than about 0.02% oxygen, or less than about 0.03% oxygen, or less than about 0.04% oxygen.

The process of the invention involves step a) wherein droplets of a suspension comprising said microorganism are formed. Accordingly, the apparatus of the present invention comprises means for formation of droplets, and more specifically an atomizing device, such as a spray nozzle. In certain examples of the invention, the atomizing device (spray nozzle) is selected from the group consisting of a two-fluid nozzle (e.g. using nitrogen or other inert gases such as noble gases as atomizing gas), an ultrasound nozzle, a pressure nozzle, a vibrating nozzle, a frequency nozzle, an electrostatic nozzle, or a rotating atomizing device. In a more specific example of the invention, the atomizing device (spray nozzle) is selected from the group consisting of a two-fluid nozzle and an electrostatic nozzle.

In some examples of the invention, the atomizing means (e.g. two-fluid nozzle) comprises an inlet for a spray gas, and optionally means for controlling the pressure of the inlet spray gas.

Process step d) involves the separation of frozen particles from cryogenic material (e.g. liquid nitrogen). Thus, in some examples of the invention, the apparatus accordingly comprises means for collecting the frozen particles or separating the frozen particles from cryogenic material (such as liquid nitrogen), e.g. a sieve or a filter (e.g. and electrostatic filter).

In certain examples of the invention, the apparatus comprises a sieve, such as a sieve having an aperture diameter below about 500 micrometer, such as in the range between about 40 micrometer to about 300 micrometer, such as in the range between about 50 micrometer to about 250 micrometer, such as about 50 micrometer, such as about 100 micrometer, such as about 150 micrometer, such as about 200 micrometer, or such as about 250 micrometer.

In a more specific example of the invention, the means for collecting the frozen particles is a sieve, such as a sieve having an aperture diameter in the range from about 40 micrometer to about 300 micrometer.

In further examples of the present disclosure, the cryogenic material is selected from the group of helium, hydrogen, nitrogen, air, fluorine, argon, oxygen, methane, carbon dioxide, nitrous oxide and/or nitrous carbon. The cryogenic material may be in gaseous phase and/or liquid phase, thus the cryogenic material may be one or more cryogenic liquids and/or one or more cryogenic gases. The cryogenic material has a boiling point below −50° C. (−58° F.), and typically below −150° C. (−238° F.), at 1 atm pressure. In a specific example of the present disclosure, the cryogenic material is liquid nitrogen. In some examples of the present disclosure, the cryogenic material has a temperature of −50° C. (−58° F.) or lower, more preferably −75° C. (−103° F.) or lower, yet more preferably −100° C. (−148° F.) or lower, even yet more preferably −125° C. (−193° F.) or lower, most preferably −150° C. (−238° F.) or lower.

### NUMBERED ITEMS FURTHER DESCRIBING THE INVENTION

1. A process for preserving bacteria in a suspension comprising strict anaerobic bacteria, the process comprising the following steps:
a) forming droplets of a liquid suspension comprising said strict anaerobic bacteria by spraying or atomizing the suspension,
b) discharging the droplets into a chamber comprising cryogenic material, such as liquid nitrogen, to produce frozen particles; and
c) separating the frozen particles obtained in b) from the cryogenic material, such as liquid nitrogen, to obtain purified frozen particles.

2. A process for preserving bacteria in a suspension comprising strict anaerobic bacteria, the process comprising the following steps:
a) forming droplets of a liquid suspension comprising said strict anaerobic bacteria by spraying or atomizing the suspension
b) contacting the droplets with cryogenic material, such as liquid nitrogen, and/or a cryogenic gas, to produce frozen particles; and
c) separating the frozen particles obtained in b) from the cryogenic material, such as liquid nitrogen, to obtain purified frozen particles
wherein the process steps a) to c) are performed in the presence of no more than about 2% oxygen, for example less than about 1% oxygen, preferably less than about 0.5% oxygen, e.g. less than about 0.05% oxygen.

3. The process according to item 1 or 2, wherein the purified frozen particles are subjected to a drying step d), such as under reduced pressure, e.g. freeze drying, to produce dried particles.

4. The process according to item 1, wherein the frozen particles obtained from step c) or dried particles obtained in step d) are packaged in a package step e) in an air-tight and/or moisture-tight package.

5. The process according to item 1, wherein the steps a) to c) are performed in the presence of less than about 0.5% oxygen, such as less than about 0.25% oxygen, such as less than about 0.1% oxygen, such as less than about 0.05% (about 5 ppm) oxygen, such as less than about 0.02% oxygen, or less than about 0.03% oxygen, or less than about 0.04% oxygen, preferably wherein steps d) and e) are performed under said oxygen concentration.

6. The process according to any one of the preceding items, wherein the process steps a) to c) are performed in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%, preferably wherein steps d) and e) are also performed under said oxygen concentration.

7. The process according to any one of the preceding items, wherein the process steps a) to c) are performed in the presence of oxygen in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04% preferably wherein steps d) and e) are also performed under said oxygen concentration.

8. The process according to any one of the preceding items, wherein the steps a) to c) are performed under essentially anaerobic conditions, preferably wherein steps d) and e) are also performed under essentially anaerobic conditions.

9. The process according to any one of the preceding items, wherein steps a) to c) are performed in the presence of a gas that is not oxygen, such as for example nitrogen gas or a noble gas, preferably wherein steps d) and e) are also performed under said gas conditions.

10. The process according to any one of the preceding items, wherein the suspension comprises at least one species of strict anaerobic bacteria selected from the group consisting of Adlercreutzia sp., Akkermansia sp., Alistipes sp., Anaerotruncus sp., Bacteroidales, Bacteroides sp., Blautia sp., Butyricicoccus sp., Butyrivibrio sp., Catabacteriaceae sp., Christensenella sp., Clostridiales sp., Clostridium sp., Collinsella sp., Coprococcus sp., Cutibacterium sp., Dialister sp., Dorea sp., Erysipelotrichaceae sp. Eubacterium sp., Faecalibacterium sp., Flavonifractor sp., Fusobacterium sp., Hafnia sp., Holdemania sp., Hungatella sp., Intestinibacter sp., Lachnobacterium sp., Lachnospira sp., Lachnospiraceae sp, Lachnospiraceae gen. nov. sp. Nov, Lachnospiraceae sp. nov., Methanobrevibacter sp., Methanomassiliicoccus sp., Methanosarcina sp., Mitsuokella sp., Odoribacter sp., Oscillospira sp., Oxalobacter sp., Parabacteroides sp., Phascolarctobacterium sp., Porphyromonadaceae sp., Prevotella sp., Propionibacterium sp., Rikenellaceae sp., Roseburia sp. Ruminococcus sp., Subdoligranulum sp., Sutterella sp., Turicibacteraceae sp.

11. The process according to any one of the preceding items, wherein the suspension comprises at least one species of strict anaerobic bacteria selected from the group consisting of Adlercreutzia sp., Adlercreutzia equolifaciens, Akkermansia sp., Akkermansia muciniphila, Alistipes sp., Alistipes finegoldii, Alistipes hadrus, Alistipes indistinctus, Alistipes onkerdonkii, Alistipes putredinis Alistipes shahii, Anaerostipes sp. Anaerostipes caccae, Anaerostipes hadrus, Anaerotruncus sp., Bacteroidales, Bacteroides sp., Bacteroides dorei, Bacteroides fragilis, Bacteroides intestinalis, Bacteroides intestinihominis, Bacteroides ovatus, Bacteroides putredinis, Bacteroides thetaiotaomicron, Bacteroides uniformis, Bacteroides vulgatus, Bacteroides xylanisolvens, Blautia sp, Blautia luti, Blautia obeum, Blautia wexlerae, Butyricicoccus, Butyrivibrio fibrisolvens, Butyrivibrio sp., Catabacteriaceae, Christensenella sp., Clostridiales, Clostridium sp., Clostridium scindens, Clostridium spiroforme, Clostridium butyricum, Collinsella sp., Collinsella aerofaciens, Coprococcus sp., Coprococcus catus, Coprococcus comes, Coprococcus eutactus, Coprococcus sp., Cutibacterium acnes, Dialister sp., Dialister invisus, Dorea sp., Dorea formicigenerans, Dorea longicatena, Erysipelotrichaceae, Eubacterium sp. Eubacterium eligens, Eubacterium hallii, Eubacterium limosum, Eubacterium ramulus, Eubacterium rectale, Eubacterium siraeum, Eubacterium ventriosum, Faecalibacterium sp., Faecalibacterium prausnitzii, Flavonifractor plautii, Fusobacterium prausnitzii, Hafnia, Holdemania, Hungatella hathewayi, Intestinibacter bartlettii, Lachnobacterium, Lachnospira, Lachnospira pectinoshiza, Lachnospiraceae, Lachnospiraceae gen. nov. sp. Nov, Lachnospiraceae sp. nov., Methanobrevibacter sp., Methanomassiliicoccus sp., Methanosarcina, Mitsuokella multiacidus, Odoribacter, Oscillospira, Oxalobacter formigenes, Parabacteroides sp., Parabacteroides distasonis, Phascolarctobacterium, Porphyromonadaceae, Prevotella sp., Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella veroralis, Propionibacterium acnes, Rikenellaceae, Roseburia sp. Roseburia faecis, Roseburia hominis, Roseburia intestinalis, Roseburia inulinivorans, Ruminococcus sp., Ruminococcus bicirculans, Ruminococcus gauvreauii, Ruminococcus gnavus, Ruminococcus lactaris, Ruminococcus obeum, Ruminococcus torques, Ruminococcus albus, Ruminococcus bromii, Ruminococcus callidus, Ruminococcus flavefaciens, Ruminococcus gauvreauii, Subdoligranulum, Sutterella and Turicibacteraceae.

12. The process according to any one of the preceding items, wherein step a) is performed in a chamber having a pressure kept in the range between about 60 kPa to 200 kPa, such as in the range between 80 kPa and 120 kPa, preferably in the range between 90 kPa to about 110 kPa, e.g. at about 101 kPa (atmospheric pressure).

13. The process according to any one of the preceding items, wherein step b) is performed in a chamber having a pressure kept in the range between about 60 kPa to 200 kPa, such as in the range between 80 kPa and 120 kPa, preferably in the range between 90 kPa to about 110 kPa, e.g. at about 101 kPa (atmospheric pressure).

14. The process according to any one of the preceding items, wherein steps a), b) and c) is performed in a chamber having a pressure kept in the range between about 60 kPa to 200 kPa, such as in the range between 80 kPa and 120 kPa, preferably in the range between 90 kPa to about 110 kPa, e.g. at about 101 kPa (atmospheric pressure).

15. The process according to any one of the preceding items, wherein steps a) and b) are performed in the same chamber, e.g. by spraying the suspension into a chamber containing cryogenic material, such as liquid nitrogen.

16. The process according to any one of the preceding items, wherein steps a) and b) are performed in the same chamber, e.g. by spraying the suspension into a chamber containing cryogenic material, such as liquid nitrogen and/or nitrogen in a gas phase.

17. The process according to any one of the preceding items, wherein steps a) and b) are performed in the same chamber, e.g. by spraying the suspension into a chamber containing cryogenic material, such as liquid nitrogen, and wherein the chamber has a pressure kept in the range between 90 kPa to about 110 kPa, e.g. at about 101 kPa (atmospheric pressure).

18. The process according to any one of the preceding items, wherein the preparation of droplets is carried out by means of a spray nozzle (atomizing device), such as an ultrasound nozzle; a pressure nozzle; a two-fluid nozzle (e.g. using cryogenic gas such as N2 as atomizing gas); a vibrating nozzle; a frequency nozzle, an electrostatic nozzle; or a rotating atomizing device.

19. The process according to any one of the preceding items, wherein the preparation of droplets is carried out by means of a two-fluid nozzle.

20. The process according to any one of the preceding items, wherein the preparation of droplets is resulting in droplets having a size from about 5 to about 800 micrometer, such as from about 5 to about 600 micrometers, such as from about 5 to about 500 micrometer, such as in the range from about 5 to about 400 micrometer, such as about 10 to about 350 micrometer, about 10 micrometer to about 300 micrometer, about 10 micrometer to about 200 micrometer, such as about 10 micrometer to about 50 micrometer, or such as about 50 micrometer to about 200 micrometer, such as about 50 micrometer to about 100 micrometer, such as about 75 micrometer, or such as about 100 micrometer to about 200 micrometer, such as about 150 micrometer, measured as Dv50 values in micrometer.

21. The process according to any one of the preceding items, wherein the preparation of droplets is carried out by means of a spray nozzle (atomizing device), and the prepared droplets has a size of between 5 and 400 micrometers and preferably between 10 and 250 micrometer, measured as Dv50 values in micrometer.

22. The process according to any one of the preceding items, wherein the formation of droplets in step a) is performed using a spray gas (atomizing gas).

23. The process according to any one of the preceding items, wherein the spray gas is selected from the group consisting of an inert gas (such as Nitrogen), a noble gas (e.g. Helium, Argon or Neon), carbon dioxide, and an alkane gas (such methane), and a mixture thereof.

24. The process according to any one of the preceding items, wherein the spray gas comprises or consists of Nitrogen.

25. The process according to any one of the preceding items, wherein the droplet forming step, (e.g. the spray step) is carried out at with a spray gas inlet temperature of at most about 80° C., such as about 70° C., such as about 60° C., such as in the range between about 0° C. to about 60° C., such as in the range between about 0° C. to about 15° C., or such as in the range between about 15° C. to about 30° C., such as between about 18° C. to about 25° C., such as about 19° C., about 20° C., about 21° C., about 22° C., about 23° C., or about 24° C., such as at about 22° C. (room temperature).

26. The process according to any one of the preceding items, wherein the droplet forming step, (e.g. the spray step) is carried out at with a spray gas inlet temperature in the range between about 15° C. to about 30° C., preferably such as in the range between about 18° C. to about 25° C., such as at about 22° C.

27. The process according to any one of the preceding items, wherein the spray gas has an inlet pressure in the range between about 1 kPa to about 500 kPa, such as in the range between about 5 kPa to about 500 kPa, such as in the range between about 5 kPa to about 300 kPa, such as in the range between about 5 kPa to about 100 kPa, such as about 60 kPa, or such as about 70 kPa, or such as about 80 kPa, or such as in the range between about 100 kPa to about 400 kPa, such as about 120 kPa, or about 150 kPa, or about 200 kPa, or about 250 kPa, or about 300 kPa, or about 350 kPa.

28. The process according to any one of the preceding items, wherein the spray gas has an inlet pressure in the range between about 100 kPa to about 400 kPa.

29. The process according to any one of the preceding items, wherein the suspension further comprises one or more stabilizing additives.

30. The process according to any one of the preceding items, wherein one or more additives are added to the bacterial suspension prior to step a).

31. The process according to any one of the preceding items, wherein one or more additives are added to the suspension prior to step a) in the presence of less than about 5% oxygen, such as less than about 2% oxygen, preferably less than about 1% oxygen, such as 0.5% oxygen, such as less than about 0.05% oxygen.

32. The process according to any one of the preceding items, wherein one or more additives are added to the suspension prior to step a) in the presence of less than about 0.5% oxygen, such as less than about 0.25% oxygen, such as less than about 0.1% oxygen, such as less than about 0.05% (about 5 ppm) oxygen, such as less than about 0.02% oxygen, or less than about 0.03% oxygen, or less than about 0.04% oxygen.

33. The process according to any one of the preceding items, wherein one or more additives are added to the suspension prior to step a) in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01% oxygen, or such as about 0.02% oxygen, or in the range between about 0.025% to about 0.05% oxygen, such as about 0.03% oxygen, or such as about 0.04% oxygen.

34. The process according to any one of the preceding items, wherein one or more additives are added to the suspension prior to step a), under essentially anaerobic conditions.

35. The process according to any one of the preceding items, wherein the one or more additives is selected from the group consisting of: Inositol, lactose, sucrose, trehalose, inulin, maltodextrin, dextrose, alginate or a salt thereof (e.g. sodium alginate), skimmed milk powder, yeast extract, casein peptone, hydrolyzed protein, such as hydrolyzed casein, casein or salts thereof (such as sodium caseinate), inosine, inosinemonophospate and a salt thereof, glutamine and salts thereof (such as monosodium glutamate), ascorbic acid and salts thereof (such as sodium ascorbate), citric acid and salts thereof, polysorbate, a hydrate of Magnesium sulphate (e.g. a heptahydrate), a hydrate of Manganous sulphate (e.g. a monohydrate) and Dipotassium hydrogen phosphate, propyl gallate and a mixture thereof.

36. The process according to any one of the preceding items, wherein the one or more additive selected from the group consisting of Yeast Extract, Dextrose, Polysorbate, Dipotassium hydrogen phosphate, Magnesium sulphate heptahydrate, Manganous sulphate monohydrate.

37. The process according to any one of the preceding items, wherein the one or more additive selected from the group consisting of Yeast Extract, Dextrose, Polysorbate, Dipotassium hydrogen phosphate, Magnesium sulphate heptahydrate, Manganous sulphate monohydrate and optionally a mixture of vitamins.

38. The process according to any one of the preceding items, further comprising a concentrating step prior to step a), wherein a suspension of microorganisms (e.g. strict anaerobic bacteria) is concentrated by removing fluid from the suspension, e.g. by centrifugation or filtration.

39. The process according to any one of the preceding items, further comprising a concentrating step prior to step a), wherein a suspension of microorganisms (e.g. strict anaerobic bacteria) is concentrated by removing fluid from the suspension, e.g. by centrifugation or filtration in the presence of less than about 5% oxygen, such as less than about 2% oxygen, preferably less than about 1% oxygen, such as 0.5% oxygen, such as less than about 0.05% oxygen.

40. The process according to any one of the preceding items, wherein the concentration step is performed in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%.

41. The process according to any one of the preceding items, wherein the concentration step is performed in essentially anaerobic conditions.

42. The process according to any one of the preceding items, further comprising a fermentation step prior to step a), wherein the suspension is fermented in the presence of less than about 5% oxygen, such as less than about 2% oxygen, preferably less than about 1% oxygen, such as less than about 0.5% oxygen, such as less than about 0.05% oxygen.

43. The process according to any one of the preceding items, further comprising a fermentation step prior to step a), wherein the suspension is fermented under essentially anaerobic conditions.

44. The process according to any one of the proceedings items, further comprising a washing step prior to the droplet formation step a), wherein a suspension of microorganisms (e.g. strict anaerobic bacteria) is washed to remove components from the suspension of microorganism, e.g. components of the culture medium.

45. The process according to any one of the proceedings items, further comprising a washing step prior to the droplet formation step a), said washing step performed in the presence of less than about 5% oxygen, such as less than about 2% oxygen, preferably less than about 1% oxygen, such as 0.5% oxygen, such as less than about 0.05% oxygen.

46. The process according to any one of the proceedings items, further comprising a washing step prior to the droplet formation step a), said washing step performed in the presence of oxygen in the range between about 0.0001% to about 2% oxygen, such as in the range of about 0.0001% to about 0.5% oxygen, such as in the range between about 0.001% to about 0.05% oxygen, e.g. in the range between about 0.001% to about 0.025% oxygen, such as about 0.01%, or such as about 0.02%, or in the range between about 0.025% to about 0.05% oxygen, such as about 0.03%, or such as about 0.04%

47. The process according to any one of the proceedings items, further comprising a washing step under essentially anaerobic conditions.

48. The process according to any one of the preceding items, comprising a fermentation step, a concentration step, and the process steps a) to b), or process steps a) to c), or process steps a) to d), or process steps a) to e), which are all performed in the presence of no more than 0.5% oxygen, such as less than 0.05% oxygen.

49. The process according to any one of the preceding items, comprising a fermentation step, a concentration step, and the process steps a) to b), or process steps a) to c), or process steps a) to d), or process steps a) to e), which are all performed under essentially anaerobic conditions.

50. The process according to any one of the preceding items, comprising a fermentation step, concentration step, a washing step, and the process steps a) to b), or process steps a) to c), or process steps a) to d), or process steps a) to e), which are all performed in the presence of no more than 0.5% oxygen, such as less than 0.05% oxygen.

51. The process according to any one of the preceding items, comprising a fermentation step, concentration step, a washing step, and the process steps a) to b), or process steps a) to c), or process steps a) to d), or process steps a) to e), which are all performed under essentially anaerobic conditions.

52. The process according to any one of the preceding items, wherein the frozen particles are separated from cryogenic material, such as liquid nitrogen, using a filter (such as an electrostatic filter) or sieve.

53. The process according to any one of the preceding items, wherein the frozen particles are separated from cryogenic material, such as liquid nitrogen, and collected using a sieve, such as a sieve having an aperture diameter below about 800 micrometer, such as below about 600 micrometer, for example below about 500 micrometer, such as in the range between about micrometer to about 300 micrometer, such as in the range from about 50 micrometer to about 250 micrometer, such as about 50 micrometer, such as about 100 micrometer, such as about 150 micrometer, such as about 200 micrometer or such as about 250 micrometer.

54. The process according to any one of the preceding items, wherein the frozen particles are separated from cryogenic material, such as liquid nitrogen, using a sieve, such as a sieve having an aperture diameter in the range from about 40 micrometer to about 300 micrometer.

55. The process according to any one of the preceding items, wherein the water content of the purified frozen particles is between about 5% and about 98% by weight, such as between about 10% and about 95% by weight, (preferably between about 30% and about 80%, or between about 40% and about 75% percent by weight), with respect to the total weight of the purified frozen particles.

56. The process according to any one of the preceding items, wherein the drying of the frozen particles takes place under reduced pressure, such as by freeze-drying to produce dried particles.

57. The process according to any one of the preceding items, wherein the drying of the purified frozen particles is performed until the water activity (aw) is below about 0.8, such as below about 0.6, such as in the range of about 0.01 to 0.8, such as in the range of about 0.05 to about 0.5, such as about 0.1, or such as about 0.2, or such as about 0.3, or such as about 0.4.

58. The process according to any one of the preceding items, wherein the liquid (e.g. water) content of the dried particles is between about 0.1% and about 30% by weight, such as in the range between about 1% to about 15% by weight, such as in the range between about 5% to about 10% by weight, or such as in the range between about 0.1% to about 5% by weight, with respect to the total weight of the particles.

59. The process according to any one of the preceding items, wherein the dried particles include a microorganism having a viability of at least 1.0×10E4 per gram as defined by the most probable number (MPN).

60. The process according to any one of the preceding items, wherein the dried particles include a microorganism having a viability in the range between 1.0×10E4 to 1.0×10E13, such as in the range between about 1.0×10E4 to about 1.0×10E10 per gram, such as about 1.0×10E5, about 1×10E6, about 1.0×10E7, about 1×10E8, about 1.0×10E9, about 2.5×10E9, about 5.0×10E9, or about 7.5×10E9 per gram as defined by the most probable number (MPN).

61. A particle product comprising at least one species of strict anaerobic bacteria and being obtainable by the process of any one of the preceding items 1 to 60.

62. The particle according to item 61, further comprising one or more additives.

63. The particle according to item 61 or 62, comprising a single species of microorganism (e.g. a single species of strict anaerobic bacteria), or a plurality of species of microorganisms (e.g. a plurality of species of strict anaerobic bacteria).

64. The particle according to any of item 61 to 63, wherein the particle comprises at least one species of strict anaerobic bacteria selected from the group consisting of Adlercreutzia sp., Akkermansia sp., Alistipes sp., Anaerotruncus sp., Bacteroidales, Bacteroides sp., Blautia sp., Butyricicoccus sp., Butyrivibrio sp., Catabacteriaceae sp., Christensenella sp., Clostridiales sp., Clostridium sp., Collinsella sp., Coprococcus sp., Cutibacterium sp., Dialister sp., Dorea sp., Erysipelotrichaceae sp. Eubacterium sp., Faecalibacterium sp., Flavonifractor sp., Fusobacterium sp., Hafnia sp., Holdemania sp., Hungatella sp., Intestinibacter sp. Lachnobacterium sp., Lachnospira sp., Lachnospiraceae sp, Lachnospiraceae gen. nov. sp. Nov, Lachnospiraceae sp. nov., Methanobrevibacter sp., Methanomassiliicoccus sp., Methanosarcina sp., Mitsuokella sp., Odoribacter sp., Oscillospira sp., Oxalobacter sp., Parabacteroides sp., Phascolarctobacterium sp., Porphyromonadaceae sp., Prevotella sp., Propionibacterium sp., Rikenellaceae sp., Roseburia sp. Ruminococcus sp., Subdoligranulum sp., Sutterella sp., Turicibacteraceae sp.

65. The particle according to any of items 61 to 64, wherein the particle comprises at least one species of strict anaerobic bacteria selected from the group consisting of Adlercreutzia sp., Adlercreutzia equolifaciens, Akkermansia sp., Akkermansia muciniphila, Alistipes sp., Alistipes finegoldii, Alistipes hadrus, Alistipes indistinctus, Alistipes onkerdonkii, Alistipes putredinis Alistipes shahii, Anaerostipes sp. Anaerostipes caccae, Anaerostipes hadrus, Anaerotruncus sp., Bacteroidales, Bacteroides sp., Bacteroides dorei, Bacteroides fragilis, Bacteroides intestinalis, Bacteroides intestinihominis, Bacteroides ovatus, Bacteroides putredinis, Bacteroides thetaiotaomicron, Bacteroides uniformis, Bacteroides vulgatus, Bacteroides xylanisolvens, Blautia sp, Blautia luti, Blautia obeum, Blautia wexlerae, Butyricicoccus, Butyrivibrio fibrisolvens, Butyrivibrio sp., Catabacteriaceae, Christensenella sp., Clostridiales, Clostridium sp., Clostridium scindens, Clostridium spiroforme, Clostridium butyricum, Collinsella sp., Collinsella aerofaciens, Coprococcus sp., Coprococcus catus, Coprococcus comes, Coprococcus eutactus, Coprococcus sp., Cutibacterium acnes, Dialister sp., Dialister invisus, Dorea sp., Dorea formicigenerans, Dorea longicatena, Erysipelotrichaceae, Eubacterium sp. Eubacterium eligens, Eubacterium hallii, Eubacterium limosum, Eubacterium ramulus, Eubacterium rectale, Eubacterium siraeum, Eubacterium ventriosum, Faecalibacterium sp., Faecalibacterium prausnitzii, Flavonifractor plautii, Fusobacterium prausnitzii, Hafnia, Holdemania, Hungatella hathewayi, Intestinibacter bartlettii, Lachnobacterium, Lachnospira, Lachnospira pectinoshiza, Lachnospiraceae, Lachnospiraceae gen. nov. sp. Nov, Lachnospiraceae sp. nov., Methanobrevibacter sp., Methanomassiliicoccus sp., Methanosarcina, Mitsuokella multiacidus, Odoribacter, Oscillospira, Oxalobacter formigenes, Parabacteroides sp., Parabacteroides distasonis, Phascolarctobacterium, Porphyromonadaceae, Prevotella sp., Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella veroralis, Propionibacterium acnes, Rikenellaceae, Roseburia sp. Roseburia faecis, Roseburia hominis, Roseburia intestinalis, Roseburia inulinivorans, Ruminococcus sp., Ruminococcus bicirculans, Ruminococcus gauvreauii, Ruminococcus gnavus, Ruminococcus lactaris, Ruminococcus obeum, Ruminococcus torques, Ruminococcus albus, Ruminococcus bromii, Ruminococcus callidus, Ruminococcus flavefaciens, Ruminococcus gauvreauii, Subdoligranulum, Sutterella and Turicibacteraceae.

66. The particle according to any of items 61 to 65, wherein the suspension comprises a strict anaerobic bacteria selected from the group consisting of F. prausnitzii and E. hallii.

67. The particle according to any of items 61 to 66, wherein the one or more additive is selected from the group consisting of: Inositol, lactose, sucrose, trehalose, inulin, maltodextrin, alginate (such as sodium alginate), skimmed milk powder, yeast extract, casein peptone, inosine, inosinemonophospate, glutamine and salts thereof (such as monosodium glutaminate), casein or salts thereof (such as sodium caseinate), ascorbic acid and salts thereof (such as sodium ascorbate), propyl gallate or salts thereof, polysorbate, a hydrate of Magnesium sulphate (e.g. a heptahydrate), a hydrate of Manganous sulphate (e.g. a monohydrate) and Dipotassium hydrogen phosphate.

68. The particle according to any of items 61 to 67, wherein the particle has a size from about 5 to about 800 micrometer, such as from about 5 to about 600 micrometer, such as from about 5 to about 500 micrometer, such as in the range from about 5 to about 400 micrometer, such as about 10 to about 350 micrometer, about 10 micrometer to about 300 micrometer, about 10 micrometer to about 250 micrometer, such as about 10 micrometer to about 50 micrometer, or such as about 50 micrometer to about 200 micrometer, such as about 50 micrometer to about 100 micrometer, such as about 75 micrometer, or such as about 100 micrometer to about 200 micrometer, such as about 150 micrometer, measured as Dv50 values in micrometer.

69. The particle according to any of items 61 to 68, wherein the particle has a size of between about 5 and about 400 micrometers, preferably between about 10 to about 250 micrometer, as measured as Dv50 values in micrometer.

70. The particle according to any of items 61 to 69, wherein the dried particles include a microorganism having a viability of at least 1*106 per gram as defined by the most probable number (MPN).

71. The particle according to any of items 61 to 70, wherein the dried particles include a microorganism having a viability above 1.0×107 per gram as defined by the most probable number (MPN), such as in the range between 1.0×106 to 1.0×109 per gram such as in the range between about 1.0×106 to about 1.0×107 per gram as defined by the most probable number (MPN).

72. The particle according to any of items 59 to 69, wherein the particle is a dried particle having a water activity (aw) below about 0.8, such as below 0.6, such as in the range of about 0.01 to about 0.8, such as in the range of about 0.05 to about 0.5, such as about 0.1, or such as about 0.2, or such as about 0.3, or such as about 0.4, preferably wherein the particle is a dried particle having a water activity (aw) below about 0.5, such as in the range of about 0.05 to about 0.5.

73. The particle according to any of items 61 to 72, wherein the particle is a dried particle comprising at least one species of strict anaerobic bacteria, and having a size of between about and about 400 micrometers, preferably between about 10 and about 200 micrometer, as measured as Dv50 values in micrometer, and further having a water activity (aw) below about 0.5, such as in the range of about 0.05 to about 0.5.

74. The particle according to any of items 61 to 73, the particle (e.g. the dried particle) having a e.g. water content between about 0.1% and about 30% by weight, such as in the range between about 1% to about 15% by weight, such as in the range between about 5% to about 10% by weight, or such as in the range between about 0.1% to about 5% by weight, with respect to the total weight of the particle.

75. The particle according to any of items 61 to 74, wherein the particle is a dried particle comprising at least one species of strict anaerobic bacteria, and having a size of between about and about 400 micrometers, preferably between about 10 micrometer to about 200 micrometer, as measured as Dv50 values in micrometer, and further having a liquid (e.g. water) content between about 0.1% and about 30% by weight, such as in the range between about 1% to about 15% by weight, such as in the range between about 5% to about 10% by weight, or such as in the range between about 0.1% to about 5% by weight, with respect to the total weight of the particle.

76. The particle according to any of items 61 to 75, wherein a plurality of said dried particles form a free-flowing powder.

77. Use of a spray freezing process for stabilization of strict anaerobic bacteria.

78. Use of a spray freeze drying process for stabilization and drying of strict anaerobic bacteria.

### DETAILED DESCRIPTION OF DRAWINGS

The invention will in the following be described in greater detail with reference to the accompanying drawings. The drawings are exemplary and are intended to illustrate some of the features of the presently disclosed methods for spray freezing and freeze-drying of microorganisms and microorganisms embedded in dried particles and are not to be construed as limiting to the presently disclosed invention.

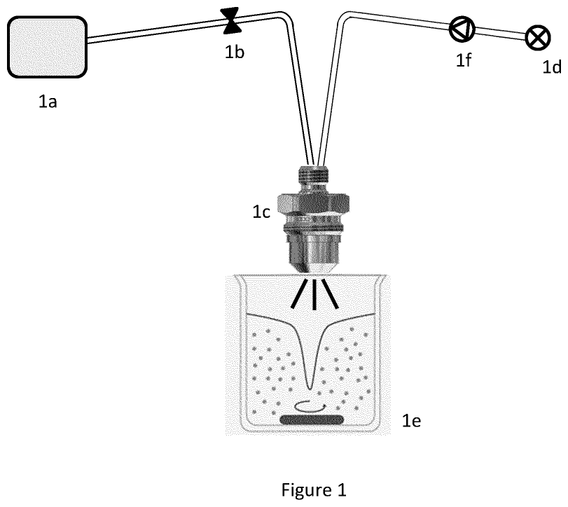

FIG. 1 shows a schematic illustration of the setup for a spray-freezing unit.

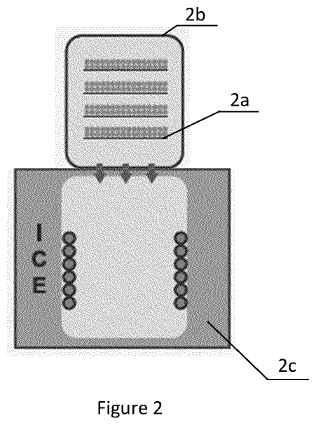

FIG. 2 shows a schematic illustration of the freeze-drying unit.

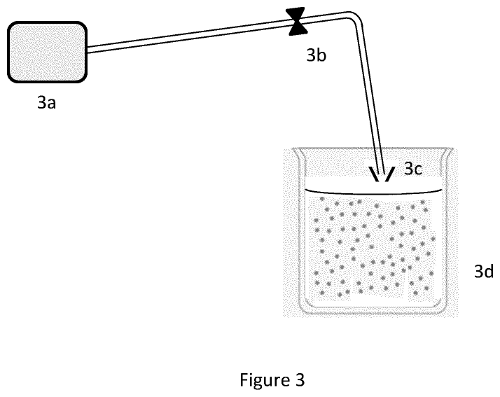

FIG. 3 shows a schematic illustration of the setup used in producing frozen pellets.

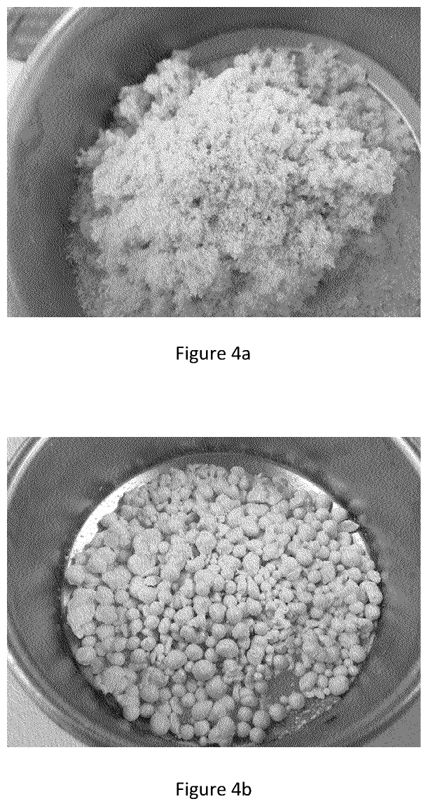

FIG. 4a-b show quantitatively the macroscopic difference in particle morphology in using either spray freezing (4a) or pelletizing (4b).

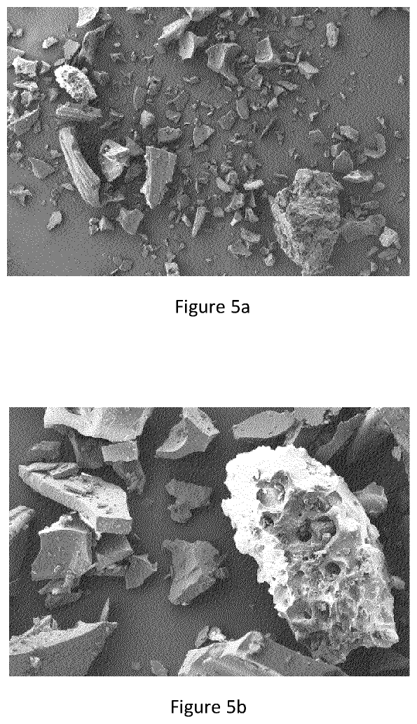

FIG. 5a-b show illustrative scanning electron micrographs of milled freeze-dried frozen pellets at 50× magnification (5a) and 250× magnification (5b). The dried particles comprise an additive and microorganisms.

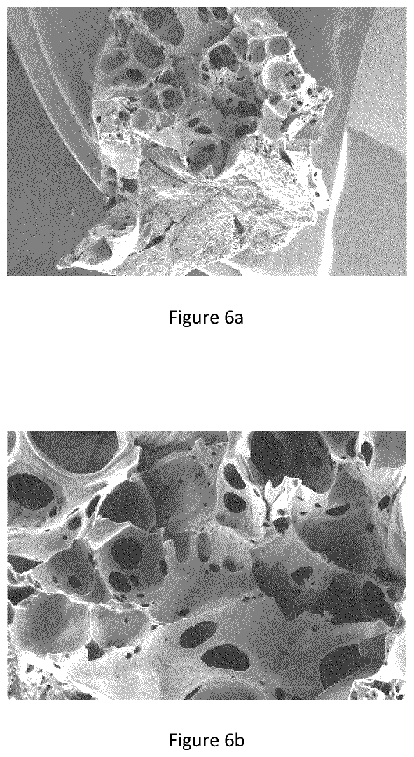

FIG. 6a-b show illustrative scanning electron micrographs of freeze-dried frozen pellets at 16× magnification (6a) and 250× magnification (6b). The dried particles comprise an additive and microorganisms.

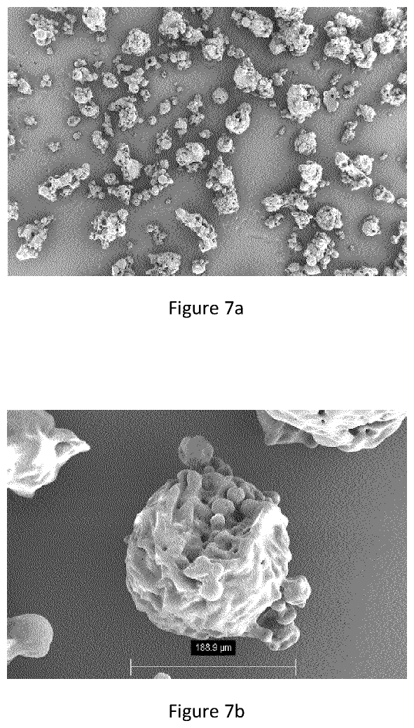

FIG. 7a-b show illustrative scanning electron micrographs of freeze-dried spray-frozen particles at 25× magnification (7a) and 250× magnification (7b). The dried particles comprise an additive and microorganisms.

### Example 1 Manufacturing of Dried Particles Comprising Faecalibacterium prausnitzii (F. prausnitzii)

The fermentation of F. prausnitzii and up-concentration using cross flow filtration was performed in a 10 liter Infors® fermenter by a standard process not unknown to those skilled in the art. Total solid content of the resulting concentrate was 12.55%. Sucrose was added as drying protectant, suitable for protecting microorganisms during cryogenic freezing, to the suspension designated for spray freezing and pelletizing. These additives were added such that the ratio between the total solid content of the concentrate and total solid content of these additives were 1:4.

Spray Freezing Method and Process Description

The liquid suspension (feed) prepared as above was pumped from a feed container (cf. FIG. 1) (1a) through a Watson-Marlow® peristaltic pump (1b) to a Spraying Systems Co.® two-fluid nozzle (1c). The liquid feed is atomized by the Spraying Systems Co.® two-fluid nozzle (1c) into a container filled with liquid nitrogen (LN₂) (1e) placed in direct succession to the Spraying Systems Co.® two-fluid nozzle outlet (1c). Flow and atomization pressure were controlled by a valve on the N₂ gas-supplying unit (1d) and by an additional valve (1f) before the inlet to the two-fluid nozzle.

Thus, the feed was atomized into LN 2 and the spray frozen product suspension was thereafter filtered through a 50 μm Retsch® filter. After the spray frozen material was separated from the LN₂ using a 50 μm Retsch® filter, the spray frozen material was loaded onto freeze-drying trays (cf. FIG. 2) (2a), placed in a vacuum chamber (2b) in connection with a cooled coils process condenser (2c) and subsequently freeze-dried.

Spray Freezing and Freeze-Drying F. prausnitzii:

The liquid suspension (feed) prepared as above, was spray frozen and freeze-dried using the procedure described above.

The liquid feed was spray frozen using a Spraying Systems Co.® two-fluid nozzle (SU2 Fluid Cap™ 2850+Air Cap™ 70). The nozzle orifice was 0.71 mm and the air cap orifice was 1.78 mm. This combination of Air Cap™ and Fluid Cap™ resulted in a spray angle of approximately 21-22°.

The atomization pressure used was 0.3 bar(g) and the feed rate was controlled by the Watson-Marlow® pump, which was set to approximately 46.2 ml/min. The spray frozen material was collected by a 50 μm sieve from Retsch®.

The collected spray frozen material (cf. FIG. 4a) was transferred to a plastic container and kept cold on dry ice until it was transferred to an anaerobic glovebox and thereafter loaded to the freeze-drier previously described (cf. FIG. 2).

The spray frozen material was evenly distributed on a metal freeze-drying tray, which was placed on the bottom shelf in the freeze-drier. After approximately 46 hours, the freeze-drying was ended, and the freeze-dried material was removed from the freeze-drying tray. The freeze-dried material was loaded to a small aluminum bag, which subsequently was welded.

Pelletizing Method and Process Description

The liquid suspension (feed) prepared as above was pumped from a feed container (cf. FIG. 3) (3a) through a Watson-Marlow® peristaltic pump (3b) to a nozzle (3c). The liquid feed was dripping from nozzle (3c) into a container filled with liquid nitrogen (LN 2) (3d) placed directly under the nozzle outlet.

The pelletized material was thereafter filtered through a 50 μm Retsch® filter, where the frozen pellets were collected. After the pelletized material was separated from the LN 2 using a 50 μm Retsch® filter, the frozen pellets were loaded onto freeze-drying trays (cf. FIG. 2) and subsequently freeze-dried as described previously.

Pelletizing and Freeze-Drying F. prausnitzii:

The liquid suspension (feed) prepared as above was pelletized and freeze-dried using the procedure described previously without atomization gas. The feed rate was controlled by the Watson-Marlow® pump, which was set to approximately 13.86 ml/min.

The pelletized material (cf. FIG. 4b) were collected by a 50 μm sieve from Retsch® and transferred to a plastic container and kept cold on dry ice until it was transferred to the anaerobic glovebox and thereafter loaded to the freeze-drier.

The pelletized material was evenly distributed on a metal freeze-drying tray, which was placed on the middle shelf in the freeze-drier. After approximately 46 hours, the freeze-drying was ended, and the freeze-dried material was removed from the freeze-drying tray. The freeze-dried material was loaded to a small aluminum bag, which subsequently was welded.

The freeze-dried frozen pellets were very inhomogeneous with respect to size, making size distribution determination impossible. Thus, the freeze-dried pellets were milled manually in a mortar for approximately 5 minutes.

Analytics and Results

All produced samples were evaluated for residual moisture (RM %), water activity (a_{w}) and particle size distribution by virtue of d₅₀ and span (freeze-dried pellets excluded from particle size evaluation).

All produced samples were likewise evaluated in terms of Viability. Viability was measured by MPN (Most Probable Number), CFU (Colony Forming Units) and flow cytometry. The reported values are mean values of 3 samples for each process type.

Size Distribution

Size distribution as measured using a Malvern Mastersizer® 3000 analytical equipment.

TABLE 1
Water activity (a_{w}) at room temperature, Residual moisture (RM
%), mean particle size distribution (d₅₀) and span of F. prausnitzii
produced by spray freezing followed by freeze-drying, freeze-dried
pellets and milled freeze dried pellets.

| Sample | a_{w} | RM % | d₅₀ | Span |
|---|---|---|---|---|
| Spray freezing + freeze- | 0.050 | 0.47 | 486 μm | 2.345 |
| drying | | | | |
| Freeze dried pellets | 0.078 | 0.40 | — | — |
| Milled freeze dried pellets | 0.074 | 0.94 | 833 μm | 2.547 |

Most Probable Number (MPN)

Growth was measured on the fermentate (FM), concentrate, concentrate+cryoprotective, frozen product (spray frozen and pellets) and freeze-dried material.

The cell count pr. ml or cell count pr. gram of all the samples is an average of six analytical results.

TABLE 2
MPN results of F. prausnitzii analysis.

| | MPN | | | |
|---|---|---|---|---|
| | pr. mL | | 95% confidence limits | |
| Process step | or pr. g | Log₁₀ MPN | Lower | Upper |
| FM | 2.9 · 10⁹ | 9.5 | 1.1 · 10⁹ | 7.9 · 10⁹ |
| Concentrate | 1.2 · 10¹¹ | 11.0 | 4.2 · 10¹⁰ | 3.3 · 10¹¹ |
| Conc. + cryo. | 1.3 · 10⁹ | 9.1 | 5.2 · 10⁸ | 3.3 · 10⁹ |
| Spray frozen (frozen) | 3.9 · 10⁸ | 8.6 | 1.4 · 10⁸ | 1.1 · 10⁹ |
| Frozen pellets | 1.7 · 10⁸ | 8.2 | 6.6 · 10⁷ | 4.6 · 10⁸ |
| Spray freeze-dried | 5.2 · 10⁷ | 7.7 | 2.0 · 10⁷ | 1.6 · 10⁸ |
| Freeze-dried pellets | 3.7 · 10⁸ | 8.6 | 1.5 · 10⁸ | 1.1 · 10⁹ |
| Milled freeze dried | 5.3 · 10⁷ | 7.7 | 2.0 · 10⁷ | 1.7 · 10⁸ |
| pellets | | | | |

From the MPN results it can be seen that the viability decreases 70% during spray freezing and the viability decreases 87% during pelletizing.

From the MPN results it is seen that the viability decreases 69% during the milling step.

Colony Forming Units (CFU)

CFU was measured on the fermentate (FM), concentrate, concentrate+cryoprotective, frozen product (spray frozen and pellets) and freeze-dried material.

TABLE 3
CFU analyses of the different process steps.

| Process step | CFU count (cells/mL) | Log |
|---|---|---|
| FM | 3.07 · 10⁸ | 8.5 |
| Concentrate | 1.02 · 10⁹ | 9.0 |
| Concentrate + cryo. | 4.97 · 10⁷ | 7.7 |
| Spray freeze (frozen) | 8.97 · 10⁶ | 7.0 |
| Pellets (frozen) | 8.27 · 10⁶ | 6.9 |
| Spray freeze-dried | 1.28 · 10⁶ | 6.1 |
| Freeze-dried pellets | 1.86 · 10⁶ | 6.3 |
| Milled freeze-dried pellets | 6.90 · 10⁵ | 5.8 |

As it is seen from the CFU results, CFU/mL decreases respectively 82% during spray freezing and 83% during pelletizing.

During milling of the pellets there is a 63% decrease in CFU/mL, which also correlates with the results seen from the MPN analysis.

Flow Cytometry

Flow cytometry was measured on the fermentate (FM), frozen product (spray frozen and pellets) and freeze-dried material.

TABLE 4
Flowcytometry results of the different process steps.

| | Damaged | Intermediate | Intact | Total | |
|---|---|---|---|---|---|
| Sample | cells/ml or cells/g | | | | Intact % |
| Fermentate (FM) | 1.46 · 10⁸ | 1.63 · 10⁸ | 1.50 · 10⁹ | 1.81 · 10⁹ | 82.9 |
| Spray frozen (frozen) | 3.34 · 10¹⁰ | 3.74 · 10⁹ | 7.82 · 10⁸ | 3.79 · 10¹⁰ | 2.1 |
| Pellets (frozen) | 2.84 · 10¹⁰ | 5.58 · 10⁹ | 7.48 · 10⁸ | 3.47 · 10¹⁰ | 2.2 |
| Spray freeze-dried | 8.46 · 10¹⁰ | 5.50 · 10⁹ | 5.50 · 10⁸ | 9.07 · 10¹⁰ | 0.61 |
| Freeze-dried pellets | 8.05 · 10¹⁰ | 9.00 · 10⁸ | 4.00 · 10⁸ | 8.18 · 10¹⁰ | 0.49 |
| Milled freeze dried | 9.12 · 10¹⁰ | 8.16 · 10⁸ | 2.04 · 10⁸ | 9.22 · 10¹⁰ | 0.21 |
| pellets | | | | | |

As it is seen from the flow cytometry results, there was a high number of intact F. prausnitzii cells in all the analyzed samples.

It is seen from the above table that the number of total cells/g in the dried powders are comparable for the freeze-dried and milled powders.

The spray freeze-dried powder has the highest number of intact cells/g.

It is seen that the viability decreases 49% when the freeze-dried pellets are milled. This is in correlation with what was also observed in the MPN and CFU analysis.

SEM Images

FIGS. 5-7 show scanning electron micrographs of dried particles from each process step of example 1 as described elsewhere herein. The particles thereby comprise an additive (sucrose) as a drying protectant, and microorganisms.

FIG. 5a-b show illustrative scanning electron micrographs of milled freeze-dried frozen pellets at 50× magnification (5a) and 250× magnification (5b). The dried particles comprise an additive (sucrose) and microorganisms. FIG. 6a-b show illustrative scanning electron micrographs of freeze-dried frozen pellets at 16× magnification (6a) and 250× magnification (6b). The dried particles comprise an additive (sucrose) and microorganisms. FIG. 7a-b show illustrative scanning electron micrographs of freeze-dried spray-frozen particles at 25× magnification (7a) and 250× magnification (7b). The dried particles comprise an additive (sucrose) and microorganisms.

When consulting detailed description of drawings it is evident that the freeze-dried spray frozen particles demonstrate the closest resemblance to a sphere with a smooth surface.

### Example 2 Manufacturing of Dried Particles Comprising Akkermansia Muciniphila (A. Muciniphila)

The fermentation of A. muciniphila and up-concentration using cross flow filtration was performed in two separate 10 liter Infors® fermenters by a standard process not unknown to those skilled in the art. The products were mixed and the total solid content of the resulting concentrate was 9.85%. Sucrose was added to the concentrate as cryoprotectant, suitable for protecting microorganisms during cryogenic freezing, thereby forming the solution that was subsequently used for spray freezing and pelletizing. These additives were added such that the ratio between the total solid content of the concentrate and total solid content of these additives was 1:4.

Spray Freezing Method and Process Description

The liquid suspension (feed) prepared as above was pumped from a feed container (cf. FIG. 1) (1a) through a Watson-Marlow® peristaltic pump (1b) to a Spraying Systems Co.® two-fluid nozzle (1c). The liquid feed is atomized by the Spraying Systems Co.® two-fluid nozzle (1c) into a container filled with liquid nitrogen (LN 2) (le) placed in direct succession to the Spraying Systems Co.® two-fluid nozzle outlet (1c). Flow and atomization pressure were controlled by a valve on the N₂ gas-supplying unit (1d) and by an additional valve (1f) before the inlet to the two-fluid nozzle.

Thus, the feed was atomized into LN₂ and the spray frozen product suspension was thereafter filtered through a 50 μm Retsch® filter. After the spray frozen material was separated from the LN₂ using a 50 μm Retsch® filter, the spray frozen material was loaded onto freeze-drying trays (cf. FIG. 2) (2a), placed in a vacuum chamber (2b) in connection with a cooled coils process condenser (2c) and subsequently freeze-dried.

Spray Freezing and Freeze-Drying A. Muciniphila

The liquid suspension (feed) prepared as above, was spray frozen and freeze-dried using the procedure described above.

The liquid feed was spray frozen using a Spraying Systems Co.® two-fluid nozzle (SU2 Fluid Cap™ 2850+Air Cap™ 70). The nozzle orifice was 0.71 mm and the air cap orifice were 1.78 mm. This combination of Air Cap™ and Fluid Cap™ resulted in a spray angle of approximately 21-22°.

The atomization pressure used was 0.3 bar(g) and the feed rate was controlled by the Watson-Marlow® pump, which was set to approximately 46.2 ml/min. The spray frozen material was collected by a 50 μm sieve from Retsch®.

The collected spray frozen material was transferred to a plastic container and kept cold on dry ice until it was transferred to an anaerobic glovebox and thereafter loaded to the freeze-drier previously described (cf. FIG. 2).

The spray frozen material was evenly distributed on a metal freeze-drying tray, which was placed on the bottom shelf in the freeze-drier. After approximately 46 hours, the freeze-drying was ended, and the freeze-dried material was removed from the freeze-drying tray. The freeze-dried material was loaded to a small aluminum bag, which subsequently was welded.

Pelletizing Method and Process Description

The liquid suspension (feed) prepared as above was pumped from a feed container (cf. FIG. 3) (3a) through a Watson-Marlow® peristaltic pump (3b) to a nozzle (3c). The liquid feed was dripping from the nozzle (3c) into a container filled with liquid nitrogen (LN₂) (3d) placed directly under the nozzle outlet.

The pelletized material was thereafter filtered through a 50 μm Retsch® filter, where the frozen pellets were collected. After the pelletized material was separated from the LN 2 using a 50 μm Retsch® filter, the frozen pellets were loaded onto freeze-drying trays (cf. FIG. 2) subsequently freeze-dried as described previously.

Pelletizing and Freeze-Drying A. Muciniphila

The liquid suspension (feed) prepared as above was pelletized and freeze-dried using the procedure described previously without atomization gas. The feed rate was controlled by a Watson-Marlow® pump, which was set to approximately 13.86 ml/min.

The pelletized material was collected by a 50 μm sieve from Retsch® and transferred to a plastic container and kept cold on dry ice until it was transferred to the anaerobic glovebox and thereafter loaded to the freeze-drier.

The pelletized material was evenly distributed on a metal freeze-drying tray, which was placed on the middle shelf in the freeze-drier. After approximately 46 hours, the freeze-drying was ended, and the freeze-dried material was removed from the freeze-drying tray. The freeze-dried material was loaded to a small aluminum bag, which subsequently was welded.

The freeze-dried frozen pellets were very inhomogeneous with respect to size, making size distribution determination impossible. Thus, the freeze-dried pellets were milled manually in a mortar for approximately 5 minutes.

Analytics and Results

All produced samples were evaluated for residual moisture (RM %), water activity (a w) and particle size distribution by virtue of d₅₀ and span (freeze-dried pellets excluded from particle size evaluation).

All produced samples were likewise evaluated in terms of Viability. Viability was measured by MPN (Most Probable Number) and flow cytometry. The reported values are mean values of 3 samples for each process type.

Size Distribution

Size distribution as measured using a Malvern Mastersizer® 3000 analytical equipment.

TABLE 5
Water activity (a_{w}) at room temperature, Residual moisture
(RM %), mean particle size distribution (d₅₀) and span
of A. muciniphila produced by spray freezing followed by
freeze-drying, freeze-dried pellets and milled pellets.

| Sample | a_{w} | RM % | d₅₀ | Span |
|---|---|---|---|---|
| Spray freezing + freeze | 0.082 | 0.82 | 269 μm | 2.475 |
| drying | | | | |
| Freeze dried pellets | 0.089 | 0.25 | — | — |
| Milled freeze dried pellets | 0.206 | 1.25 | 808 μm | 2.633 |

Most Probable Number (MPN)

Growth was measured on the fermentate, concentrate, concentrate+cryoprotectant, frozen product (spray frozen and pellets) and freeze dried material.

The cell count pr. ml or cell count pr. gram of all the samples is an average of six analytical results.

TABLE 6
MPN results of A. muciniphila analysis.

| | MPN | MPN | Standard |
|---|---|---|---|
| Process step | [pr. mL or pr. g] | Log₁₀ | deviation |
| Fermenter A | 6.5 · 10⁸ | 8.8 | 1.7 · 10⁸ |
| Fermenter B | 2.9 · 10⁸ | 8.5 | 0 |
| Concentrate | 8.2 · 10⁹ | 9.9 | 4.0 · 10⁹ |
| Conc. + cryo. | 1.0 · 10¹⁰ | 10.0 | 4.2 · 10⁹ |
| Spray frozen (frozen) | 9.1 · 10¹⁰ | 11.0 | 7.0 · 10¹⁰ |
| Frozen pellets | 4.2 · 10¹⁰ | 10.6 | 1.4 · 10⁹ |
| Spray freeze dried | 3.3 · 10¹⁰ | 10.5 | 3.9 · 10⁹ |
| Freeze dried pellets | 3.1 · 10¹⁰ | 10.5 | 6.9 · 10⁹ |
| Milled freeze-dried pellets | 7.5 · 10¹⁰ | 10.9 | 3.0 · 10¹⁰ |

From the MPN results it can be seen that high viability is achieved and that there is no viability loss during the freezing step (spray freezing and pelletizing). It can also be seen that the viability of the dried powders; e.g. spray freeze dried, freeze dried pellets and milled pellets are in the same range.

Flowcytometry

Flowcytometry was measured on the fermentates, concentrate, frozen product (spray frozen and pellets) and freeze dried material.

TABLE 7
Flowcytometry results of the different process steps.

| | Damaged | Intermediate | Intact | Total | |
|---|---|---|---|---|---|
| Sample | cells/ml or cells/g | | | | Intact % |
| Fermenter A | 3.9(±1.2) · 10⁷ | 2.9(±0.2) · 10⁸ | 7.0(±0.1) · 10⁹ | 7.3(±0.1) · 10⁹ | 95.4 |
| Fermenter B | 7.8(±1.5) · 10⁷ | 1.3(±0.06) · 10⁸ | 4.1(±0.2) · 10⁹ | 4.3(±0.2) · 10⁹ | 95.2 |
| Concentrate | 1.3(±0.2) · 10⁹ | 6.8(±0.5) · 10⁹ | 1.3(±0.2) · 10¹¹ | 1.4(±0.2) · 10¹¹ | 94.3 |
| Spray frozen | 1.7(±0.2) · 10⁹ | 7.7(±0.8) · 10⁹ | 4.6(±0.3) · 10¹⁰ | 5.5(±0.4) · 10¹⁰ | 83 |
| (frozen) | | | | | |
| Pellets (frozen) | 1.0(±0.2) · 10⁹ | 9.8(±0.6) · 10⁹ | 1.1(±0.1) · 10¹¹ | 1.2(±0.1) · 10¹¹ | 90.9 |
| Spray freeze | 3.5(±0.1) · 10⁹ | 4.0(±0.3) · 10¹⁰ | 1.3(±0.04) · 10¹¹ | 1.7(±0.07) · 10¹¹ | 74.4 |
| dried | | | | | |
| Freeze dried | 2.3(±0.4) · 10⁹ | 2.5(±0.4) · 10¹⁰ | 1.4(±0.1) · 10¹¹ | 1.7(±0.1) · 10¹¹ | 84.2 |
| pellets | | | | | |
| Milled freeze | 2.5(±0.1) · 10⁹ | 2.7(±0.1) · 10¹⁰ | 1.6(±0.05) · 10¹¹ | 1.9(±0.06) · 10¹¹ | 84.7 |
| dried pellets | | | | | |

It was seen from the flowcytometry results that there was a high number of intact A. muciniphila cells in all the analyzed samples, and that the number of total cells/g in the dried powders are high with comparable numbers for all the produced powders. A small decrease of intact cells was seen during spray freezing, but not during pelletizing.

### Example 3 Manufacturing of Dried Particles Comprising Eubacterium hallii (E. Hallii)

The fermentation of E. hallii and up-concentration using cross flow filtration was performed in two separate 10 liter Infors® fermenters by a standard process not unknown to those skilled in the art. The products were mixed and the total solid content of the resulting concentrate was 8.03%. Sucrose was added to the concentrate as cryoprotectant, suitable for protecting microorganisms during cryogenic freezing, thereby forming the solution that was subsequently used for spray freezing and pelletizing. These additives were added such that the ratio between the total solid content of the concentrate and total solid content of these additives was 1:4.

Spray Freezing Method and Process Description

The liquid suspension (feed) prepared as above was pumped from a feed container (cf. FIG. 1) (1a) through a Watson-Marlow® peristaltic pump (1b) to a Spraying Systems Co.® two-fluid nozzle (1c). The liquid feed is atomized by the Spraying Systems Co.® two-fluid nozzle (1c) into a container filled with liquid nitrogen (LN₂) (1e) placed in direct succession to the Spraying Systems Co.® two-fluid nozzle outlet (1c). Flow and atomization pressure were controlled by a valve on the N₂ gas-supplying unit (1d) and by an additional valve (1f) before the inlet to the two-fluid nozzle.

Thus, the feed was atomized into LN 2 and the spray frozen product suspension was thereafter filtered through a 50 μm Retsch® filter. After the spray frozen material was separated from the LN₂ using a 50 μm Retsch® filter, the spray frozen material was loaded onto freeze-drying trays (cf. FIG. 2) (2a), placed in a vacuum chamber (2b) in connection with a cooled coils process condenser (2c) and subsequently freeze-dried.

Spray Freezing and Freeze-Drying E. Hallii

The liquid suspension (feed) prepared as above, was spray frozen and freeze-dried using the procedure described above.

The liquid feed was spray frozen using a Spraying Systems Co.® two-fluid nozzle (SU2 Fluid Cap™ 2850+Air Cap™ 70). The nozzle orifice was 0.71 mm and the air cap orifice were 1.78 mm. This combination of Air Cap™ and Fluid Cap™ resulted in a spray angle of approximately 21-22°.

The atomization pressure used was 0.3 bar(g) and the feed rate was controlled by the Watson-Marlow® pump, which was set to approximately 46.2 ml/min. The spray frozen material was collected by a 50 μm sieve from Retsch.

The collected spray frozen material (cf. FIG. 4a) was transferred to a plastic container and kept cold on dry ice until it was transferred to an anaerobic glovebox and thereafter loaded to the freeze-drier previously described (cf. FIG. 2).

The spray frozen material was evenly distributed on a metal freeze-drying tray, which was placed on the bottom shelf in the freeze-drier. After approximately 46 hours, the freeze-drying was ended, and the freeze-dried material was removed from the freeze-drying tray. The freeze-dried material was loaded to a small aluminum bag, which subsequently was welded.

Pelletizing Method and Process Description

The liquid suspension (feed) prepared as above was pumped from a feed container (cf. FIG. 3) (3a) through a Watson-Marlow® peristaltic pump (3b) to a nozzle (3c). The liquid feed was dripping from nozzle (3c) into a container filled with liquid nitrogen (LN₂) (3d) placed directly under the nozzle outlet.

The pelletized material was thereafter filtered through a 50 μm Retsch® filter, where the frozen pellets were collected. After the pelletized material was separated from the LN₂ using a 50 μm Retsch® filter, the frozen pellets were loaded onto freeze-drying trays (cf. FIG. 2) subsequently freeze-dried as described previously.

Pelletizing and Freeze-Drying E Hallii

The liquid suspension (feed) prepared as above was pelletized and freeze-dried using the procedure described previously without atomization gas. The feed rate was controlled by a Watson-Marlow® pump, which was set to approximately 13.86 ml/min.

The pelletized material was collected by a 50 μm sieve from Retsch® and transferred to an aluminum bag and kept cold on dry ice until it was transferred to an anaerobic glovebox and thereafter loaded to the freeze drier.

The pelletized material was evenly distributed on a metal freeze drying tray, which was placed on the middle shelf in the freeze drier.

After approximately 46 hours the freeze drying was ended, and the freeze dried material was removed from the freeze drying tray. The freeze dried material was loaded to a small aluminum bag, which subsequently was welded.

The freeze-dried frozen pellets were very inhomogeneous with respect to size, making size distribution determination impossible. Thus, the freeze-dried pellets were milled manually in a mortar for approximately 5 minutes.

Analytics and Results

All produced samples were evaluated for residual moisture (RM %), water activity (aw), particle size distribution by virtue of d₅₀ and span (freeze-dried pellets excluded from particle size evaluation).

All produced samples were likewise evaluated in terms of Viability. Viability was measured by MPN (Most Probable Number) and flow cytometry. The reported values are mean values of 3 samples for each process type.

Size Distribution

Size distribution as measured using a Malvern Mastersizer® 3000 analytical equipment.

TABLE 8
Water activity (aw), Residual moisture (RM %) and mean particle size
distribution (d₅₀) of E. hallii produced by spray freezing followed
by freeze drying and freeze dried pellets and milled pellets.

| Sample | a_{w} | RM % | d₅₀ | Span |
|---|---|---|---|---|
| Spray freezing + freeze | 0.050 | 0.83 | 1290 μm | 1.549 |
| drying | | | | |
| Freeze dried pellets | 0.024 | 0.44 | — | — |
| Milled freeze dried pellets | 0.075 | 1.26 | 243 μm | 1.985 |

Most Probable Number (MPN)

Growth was measured on the fermentate, concentrate, concentrate+cryoprotectant, frozen product (spray frozen and pellets) and freeze dried material.

The cell count pr. ml or cell count pr. gram of all the samples is an average of six analytical results.

TABLE 9
MPN results of E. hallii analysis.

| | | | Standard |
|---|---|---|---|
| Process step | MPN [pr. mL or pr. g] | Log₁₀ MPN | deviation |
| Fermenter A | 6.2 · 10⁸ | 8.8 | 1.3 · 10⁸ |
| Fermenter B | 2.0 · 10⁷ | 7.3 | 1.3 · 10⁷ |
| Concentrate | 1.5 · 10¹⁰ | 10.2 | 1.6 · 10¹⁰ |
| Conc. + cryo. | 4.6 · 10⁹ | 9.7 | 1.2 · 10⁹ |
| Spray frozen (frozen) | 1.3 · 10⁶ | 6.1 | 3.5 · 10⁵ |
| Frozen pellets | 8.5 · 10⁶ | 6.9 | 4.9 · 10⁶ |
| Spray freeze dried | 8.1 · 10⁶ | 6.9 | 4.0 · 10⁶ |
| Freeze dried pellets | 1.1 · 10⁶ | 6.0 | 5.6 · 10⁵ |
| Milled freeze dried | 2.0 · 10⁴ | 4.3 | 1.8 · 10⁴ |
| pellets | | | |

From the MPN results it can be seen that the viability decreases by 3.6 log during spray freezing and the viability decreases by 2.8 log during pelletizing.

It can also be seen that the spray freeze dried powder has 0.9 log higher viability compared to the freeze dried pellets and 2.6 log higher viability compared to the milled pellets.

From the results it is seen that the viability decreases 2.6 log during the milling step.

Flowcytometry

Flowcytometry was measured on the fermentate, concentrate, concentrate with cryoprotectant, freeze dried material and electrostatic spray dried material.

TABLE 10
Flowcytometry results through the process in the test.

| | Damaged | Intermediate | Intact | Total | |
|---|---|---|---|---|---|
| Sample | cells/ml or cells/g | | | | Intact % |
| Fermenter A | 2.6(±0.3) · 10⁷ | 9.1(±0.3) · 10⁷ | 3.8(±0.0) · 10⁸ | 5.0(±0.05) · 10⁸ | 76.00 |
| Fermenter B | 4.2(±0.0) · 10⁷ | 9.1(±0.0) · 10⁷ | 3.8(±0.04) · 10⁸ | 5.1(±0.04) · 10⁸ | 74.51 |
| Concentrate | 2.9(±0.3) · 10⁸ | 6.0(±0.5) · 10⁸ | 2.0(±0.2) · 10⁹ | 2.9(±0.3) · 10⁹ | 68.97 |
| Spray frozen | 4.3(±1.3) · 10⁸ | 8.5(±3.0) · 10⁸ | 3.1(±1.6) · 10⁷ | 1.3(±0.4) · 10⁹ | 2.38 |
| (frozen) | | | | | |
| Pellets | 4.3(±0.2) · 10⁸ | 1.0(±0.0) · 10⁹ | 2.8(±0.1) · 10⁷ | 1.5(±0.02) · 10⁹ | 1.87 |
| (frozen) | | | | | |
| Spray freeze | 3.3(±0.7) · 10⁹ | 4.7(±0.9) · 10⁸ | 1.3(±0.1) · 10⁸ | 4.0(±0.8) · 10⁹ | 3.25 |
| dried | | | | | |
| Freeze dried | 4.0(±0.6) · 10⁹ | 4.5(±0.4) · 10⁸ | 6.1(±1.2) · 10⁷ | 4.4(±.6) · 10⁹ | 1.39 |
| pellets | | | | | |
| Milled freeze | 8.9(±0.1) · 10⁹ | 4.5(±0.1) · 10⁸ | 9.7(±10) · 10⁶ | 9.3(±0.2) · 10⁹ | 0.10 |
| dried pellets | | | | | |

As it is seen from the flowcytometry results, there was a high number of intact E. hallii cells in all the analyzed samples.

It can also be seen from the above table that the number of total cells/g in the dried powders are comparable for all the produced powders and it is seen that the spray freeze dried powder has the highest number of intact cells/g, followed by the freeze dried pellets.

The spray freeze dried powder has 0.3 log higher viability compared to the freeze dried pellets and 1.1 log higher viability compared to the milled pellets.

There is seen a viability loss of 0.8 log during the milling of the freeze dried pellets, which was also seen in the MPN analysis.

### Example 4 Manufacturing of Dried Particles Comprising Bacteroides Thetaiotaomicron (B. Thetaiotaomicron)

The fermentation of B. thetaiotaomicron and up-concentration using cross flow filtration was performed in two separate 10 liter Infors® fermenters by a standard process not unknown to those skilled in the art. The products were mixed and the total solid content of the resulting concentrate was 11.72%. Sucrose was added to the concentrate as cryoprotectant, suitable for protecting microorganisms during cryogenic freezing, thereby forming the solution that was subsequently used for spray freezing and pelletizing. These additives were added such that the ratio between the total solid content of the concentrate and total solid content of these additives was 1:4.

Spray Freezing Method and Process Description

The liquid suspension (feed) prepared as above was pumped from a feed container (cf. FIG. 1) (1a) through a Watson-Marlow® peristaltic pump (1b) to a Spraying Systems Co.® two-fluid nozzle (1c). The liquid feed is atomized by the Spraying Systems Co.® two-fluid nozzle (1c) into a container filled with liquid nitrogen (LN₂) (1e) placed in direct succession to the Spraying Systems Co.® two-fluid nozzle outlet (1c). Flow and atomization pressure were controlled by a valve on the N₂ gas-supplying unit (1d) and by an additional valve (1f) before the inlet to the two-fluid nozzle.

Thus, the feed was atomized into LN₂ and the spray frozen product suspension was thereafter filtered through a 50 μm Retsch® filter. After the spray frozen material was separated from the LN₂ using a 50 μm Retsch® filter, the spray frozen material was loaded onto freeze-drying trays (cf. FIG. 2) (2a), placed in a vacuum chamber (2b) in connection with a cooled coils process condenser (2c) and subsequently freeze-dried.

Spray Freezing and Freeze-Drying B. Thetaiotaomicron

The liquid suspension (feed) prepared as above, was spray frozen and freeze-dried using the procedure described above.

The liquid feed was spray frozen using a Spraying Systems Co.® two-fluid nozzle (SU2 Fluid Cap™ 2850+Air Cap™ 70). The nozzle orifice was 0.71 mm and the air cap orifice were 1.78 mm. This combination of Air Cap™ and Fluid Cap™ resulted in a spray angle of approximately 21-22°.

The atomization pressure used was 0.3 bar(g) and the feed rate was controlled by the Watson-Marlow® pump, which was set to approximately 46.2 ml/min. The spray frozen material was collected by a 50 μm sieve from Retsch®.

The collected spray frozen material (cf. FIG. 4a) was transferred to a plastic container and kept cold on dry ice until it was transferred to an anaerobic glovebox and thereafter loaded to the freeze-drier previously described (cf. FIG. 2).

The spray frozen material was evenly distributed on a metal freeze-drying tray, which was placed on the bottom shelf in the freeze-drier. After approximately 46 hours, the freeze-drying was ended, and the freeze-dried material was removed from the freeze-drying tray. The freeze-dried material was loaded to a small aluminum bag, which subsequently was welded.

Pelletizing Method and Process Description

The liquid suspension (feed) prepared as above was pumped from a feed container (cf. FIG. 3) (3a) through a Watson-Marlow® peristaltic pump (3b) to a nozzle (3c). The liquid feed was dripping from the nozzle (3c) into a container filled with liquid nitrogen (LN₂) (3d) placed under the nozzle outlet.

The pelletized material was thereafter filtered through a 50 μm Retsch® filter, where the frozen pellets were collected.

After the pelletized material was separated from the LN₂ by a 50 μm Retsch® filter, the frozen pellets were loaded onto freeze-drying trays and subsequently freeze dried as described previously.

Pelletizing and Freeze Drying B. Thetaiotaomicron

The liquid suspension (feed) prepared as above was pelletized and freeze-dried using the procedure described previously without atomization gas. The feed rate was controlled by the Watson-Marlow Pump®, which was set to approximately 13.86 ml/min. The pelletized material was collected by a 50 μm Retsch® sieve.

The pelletized material was transferred to an aluminum bag and kept cold on dry ice until it was transferred to the anaerobic glovebox and thereafter loaded to the freeze drier.

After approximately 46 hours the freeze drying was ended, and the freeze dried material was removed from the freeze drying tray.

The freeze dried material was loaded to a small aluminum bag, which subsequently was welded.

The freeze-dried frozen pellets were very inhomogeneous with respect to size, making size distribution determination impossible. Thus, the freeze-dried pellets were milled manually in a mortar for approximately 5 minutes.

Analytics

All produced samples were evaluated for residual moisture (RM %), water activity (aw), particle size distribution by virtue of d50 and span (freeze-dried pellets excluded from particle size evaluation).

All produced samples were likewise evaluated in terms of Viability. Viability was measured by MPN (Most Probable Number), CFU (Colony Forming Units) and flow cytometry. The reported values are mean values of 3 samples for each process type.

Size Distribution

Size distribution as measured using a Malvern Mastersizer® 3000 analytical equipment.

TABLE 11
Water activity (aw), Residual moisture (RM %) and mean particle
size distribution (d₅₀) of B. thetaiotaomicron produced
by spray freezing followed by freeze drying and freeze
dried pellets and milled freeze-dried pellets.

| Sample | a_{w} | RM % | d₅₀ | Span |
|---|---|---|---|---|
| Spray freezing + freeze | 0.135 | 0.83 | 396 μm | 3.252 |
| drying | | | | |
| Freeze dried pellets | 0.131 | 0.21 | — | — |
| Milled freeze dried pellets | 0.216 | 1.18 | 195 μm | 3.880 |

Most Probable Number (MPN)

Growth was measured on the fermentate, concentrate, concentrate+cryoprotectant, frozen product (spray frozen and pellets) and freeze dried material.

The cell count pr. ml or cell count pr. gram of all the samples is an average of six analytical results.

TABLE 12
MPN results of test with B. thetaiotaomicron

| | | | Standard |
|---|---|---|---|
| Process step | MPN [pr. mL or pr. g] | Log₁₀ MPN | deviation |
| Fermenter A | 2.8 · 10⁹ | 9.4 | 1.4 · 10⁸ |
| Fermenter B | 1.5 · 10⁹ | 9.2 | 2.0 · 10⁹ |
| Concentrate | 5.0 · 10¹⁰ | 10.7 | 4.0 · 10¹⁰ |
| Conc. + cryo. | 1.3 · 10¹⁰ | 10.1 | 5.1 · 10⁹ |
| Spray frozen (frozen) | 2.5 · 10⁸ | 8.4 | 2.1 · 10⁸ |
| Frozen pellets | 5.5 · 10⁶ | 6.7 | 6.4 · 10⁶ |
| Spray freeze dried | 6.3 · 10³ | 3.8 | 1.3 · 10³ |
| Freeze dried pellets | 1.6 · 10⁵ | 5.2 | 3.4 · 10⁴ |
| Milled freeze dried | 5.1 · 10⁴ | 4.7 | 1.9 · 10⁴ |
| pellets | | | |

From the MPN results it was seen that the viability of B. thetaiotaomicron decreases during the freezing step; the viability is decreased by 1.7 log during spray freezing and 3.4 log during pelletizing.

During the freeze drying step, the viability of the spray frozen material was reduced 4.6 log and the viability of the pellets was reduced 1.5 log during freeze drying.

Flowcytometry

Flowcytometry was measured on the fermentate, concentrate, frozen product (spray frozen and pellets) and freeze dried material.

TABLE 13
Flowcytometry results of the different process steps.

| | Damaged | Intermediate | Intact | Total | Intact |
|---|---|---|---|---|---|
| Sample | cells/ml or cells/g | | | | % |
| Fermenter A | 1.7(±0.03) · 10⁹ | 1.5(±0.03) · 10⁸ | 2.1(±0.03) · 10⁹ | 4.0(±0.01) · 10⁹ | 52.1 |
| Fermenter B | 1.9(±0.07) · 10⁹ | 1.9(±0.03) · 10⁸ | 1.9(±0.05) · 10⁹ | 4.1(±0.11) · 10⁹ | 47.6 |
| Concentrate | 5.6(±0.11) · 10¹⁰ | 1.4(±0.04) · 10¹⁰ | 4.0(±0.07) · 10¹⁰ | 1.1(±0.02) · 10¹¹ | 36.2 |
| Spray frozen | 1.0(±0.07) · 10¹¹ | 7.7(±0.3) · 10⁹ | 3.9(±0.08) · 10⁹ | 1.2(±0.07) · 10¹¹ | 3.4 |
| (frozen) | | | | | |
| Pellets | 9.5(±1.00) · 10¹⁰ | 4.7(±0.50) · 10⁹ | 2.7(±0.30) · 10⁹ | 1.0(±0.10) · 10¹¹ | 2.7 |
| (frozen) | | | | | |
| Spray freeze | 1.1(±1.24) · 10¹¹ | 3.7(±3.93) · 10⁹ | 2.8(±3.78) · 10⁸ | 1.1(±1.24) · 10¹¹ | 0.18 |
| dried | | | | | |
| Freeze dried | 1.1(±0.97) · 10¹¹ | 2.7(±2.40) · 10⁹ | 2.4(±3.14) · 10⁸ | 1.1(±0.96) · 10¹¹ | 0.15 |
| pellets | | | | | |
| Milled freeze | 9.1(±9.76) · 10¹⁰ | 3.9(±4.33) · 10⁹ | 2.5(±3.35) · 10⁸ | 9.2(±9.69) · 10¹⁰ | 0.17 |
| dried pellets | | | | | |

As it is seen from the flowcytometry results, there was a high number of total B. thetaiotaomicron cells in all the analyzed samples.

It is seen from the above table that the number of total cells/g through the whole process are quite high and that the total number of cells/g are in the same range for all the analyzed samples through the full process.

Like the MPN results it is seen that there is a viability decrease during the freezing step.

It is seen that during spray freezing the viability is decreased 1.0 log and during pelletizing the viability is decreased 1.2 log.

It is also seen that there is a viability decrease during freeze drying of the spray frozen material and the pellets.

The viability of the spray frozen material is decreased 1.1 log and the viability of the pellets is decreased 1.0 log during freeze drying.

The spray freeze dried powder has the highest viability and the viability is 0.1 LOG higher compared to the freeze dried pellets and the milled pellets.

### REFERENCES

WO2016083617
U.S. Pat. No. 7,007,406
Jae-Young Her, Min Suk Kim, Kwang-Geun Lee, Preparation of probiotic powder by the spray freeze-drying method, Journal of Food Engineering, Volume 150, 2015.
Ishwarya S, Anandharamakrishnan C, Stapley A. Review: Spray-freeze-drying: A novel process for the drying of foods and bioproducts. Trends In Food Science & Technology [serial online]. Feb. 1, 2015; 41:161-181.
Semyonov D, Ramon O, Kaplun Z, Levin-Brener L, Gurevich N, Shimoni E. Microencapsulation of Lactobacillus paracasei by spray freeze drying. Food Research International [serial online]. Jan. 1, 2010; 43:193-202.
Volkert M, Ananta E, Luscher C, Knorr D. Effect of air freezing, spray freezing, and pressure shift freezing on membrane integrity and viability of Lactobacillus rhamnosus GG. Journal Of Food Engineering. Jan. 1, 2008; 87:532-540.
ISO 13320:2009 standard for Particle size analysis—Laser diffraction methods

1. A process for preserving strict anaerobic bacteria in a suspension, comprising:
(a) forming droplets of a liquid suspension comprising said strict anaerobic bacteria by spraying or atomizing the suspension,
(b) discharging the droplets into a chamber comprising cryogenic material to produce frozen particles in cryogenic material, and
(c) separating the frozen particles obtained in (b) from the cryogenic material to obtain purified frozen particles,
wherein steps (a) to (c) are performed in the presence of less than 2% oxygen.

2. The process according to claim 1, further comprising:
(d) drying the purified frozen particles to produce dried particles.

3. The process according to claim 1, wherein in step (b) the cryogenic material has a temperature of −50° C. (−58° F.) or lower.

4. The process according to claim 1, wherein the cryogenic material is one or more selected from helium, hydrogen, nitrogen, air, fluorine, argon, oxygen, methane, liquid natural gas, carbon dioxide, nitrous oxide, and nitrous carbon.

5. The process according to claim 1, further comprising packaging the purified frozen particles obtained at step (c) in a package that is one or both of air-tight and moisture-tight.

6. The process according to claim 1, wherein steps (a) to (c) are performed in the presence of less than about 0.5% oxygen.

7. The process according to claim 1, wherein steps (a) to (c) are performed in the presence of a gas selected from nitrogen gas, a noble gas, carbon dioxide gas, and an alkane gas.

8. The process according to claim 1, wherein the suspension comprises at least one species of strict anaerobic bacteria selected from the group consisting of Adlercreutzia sp., Adlercreutzia equolifaciens, Akkermansia sp., Akkermansia muciniphila, Alistipes sp., Alistipes finegoldii, Alistipes hadrus, Alistipes indistinctus, Alistipes onkerdonkii, Alistipes putredinis Alistipes shahii, Anaerostipes sp. Anaerostipes caccae, Anaerostipes hadrus, Anaerotruncus sp., Bacteroidales, Bacteroides sp., Bacteroides dorei, Bacteroides fragilis, Bacteroides intestinalis, Bacteroides intestinihominis, Bacteroides ovatus, Bacteroides putredinis, Bacteroides thetaiotaomicron, Bacteroides uniformis, Bacteroides vulgatus, Bacteroides xylanisolvens, Blautia sp, Blautia luti, Blautia obeum, Blautia wexlerae, Butyricicoccus, Butyrivibrio fibrisolvens, Butyrivibrio sp., Catabacteriaceae, Christensenella sp., Clostridiales, Clostridium sp., Clostridium scindens, Clostridium spiroforme, Clostridium butyricum, Collinsella sp., Collinsella aerofaciens, Coprococcus sp., Coprococcus catus, Coprococcus comes, Coprococcus eutactus, Coprococcus sp., Cutibacterium acnes, Dialister sp., Dialister invisus, Dorea sp., Dorea formicigenerans, Dorea longicatena, Erysipelotrichaceae, Eubacterium sp. Eubacterium eligens, Eubacterium hallii, Eubacterium limosum, Eubacterium ramulus, Eubacterium rectale, Eubacterium siraeum, Eubacterium ventriosum, Faecalibacterium sp., Faecalibacterium prausnitzii, Flavonifractor plautii, Fusobacterium prausnitzii, Hafnia, Holdemania, Hungatella hathewayi, Intestinibacter bartlettii, Lachnobacterium, Lachnospira, Lachnospira pectinoshiza, Lachnospiraceae, Lachnospiraceae gen. nov. sp. Nov, Lachnospiraceae sp. nov., Methanobrevibacter sp., Methanomassiliicoccus sp., Methanosarcina, Mitsuokella multiacidus, Odoribacter, Oscillospira, Oxalobacter formigenes, Parabacteroides sp., Parabacteroides distasonis, Phascolarctobacterium, Porphyromonadaceae, Prevotella sp., Prevotella albensis, Prevotella amnii, Prevotella bergensis, Prevotella bivia, Prevotella brevis, Prevotella bryantii, Prevotella buccae, Prevotella buccalis, Prevotella copri, Prevotella dentalis, Prevotella denticola, Prevotella disiens, Prevotella histicola, Prevotella intermedia, Prevotella maculosa, Prevotella marshii, Prevotella melaninogenica, Prevotella micans, Prevotella multiformis, Prevotella nigrescens, Prevotella oralis, Prevotella oris, Prevotella oulorum, Prevotella pallens, Prevotella salivae, Prevotella stercorea, Prevotella tannerae, Prevotella timonensis, Prevotella veroralis, Propionibacterium acnes, Rikenellaceae, Roseburia sp. Roseburia faecis, Roseburia hominis, Roseburia intestinalis, Roseburia inulinivorans, Ruminococcus sp., Ruminococcus bicirculans, Ruminococcus gauvreauii, Ruminococcus gnavus, Ruminococcus lactaris, Ruminococcus obeum, Ruminococcus torques, Ruminococcus albus, Ruminococcus bromii, Ruminococcus callidus, Ruminococcus flavefaciens, Ruminococcus gauvreauii, Subdoligranulum, Sutterella and Turicibacteraceae.

9. The process according to claim 1, wherein steps (a) and (b) are performed in the same chamber by spraying the suspension into a chamber containing the cryogenic material.

10. The process according to claim 1, wherein the forming of droplets in step (a) is carried out with a two-fluid nozzle.

11. The process according to claim 1, wherein the forming of droplets in step (a) is carried out with an atomizing gas.

12. The process according to claim 1, wherein the atomizing gas is one or more selected from the group consisting of an inert gas, a noble gas, carbon dioxide, and an alkane gas.

13. The process according to claim 1, wherein the suspension further comprises one or more stabilizing additives selected from the group consisting of: inositol, lactose, sucrose, trehalose, inulin, maltodextrin, dextrose, alginate or a salt thereof, skimmed milk powder, yeast extract, casein peptone, hydrolyzed casein, casein, casein salts thereof, inosine, inosinemonophospate and salts thereof, glutamine and salts thereof, ascorbic acid and salts thereof, citric acid and salts thereof, polysorbate, a hydrate of magnesium sulphate, a hydrate of manganous sulphate (e.g. a monohydrate), dipotassium hydrogen phosphate, and propyl gallate.

14. The process according to claim 1, wherein the frozen particles are separated from cryogenic material and collected using a sieve having an aperture diameter below about 800 micrometer.

15. The process according to claim 1, wherein the water content of the purified frozen particles is between about 5% and about 98% by weight.

16. The process according to claim 2, wherein the drying of the purified frozen particles is performed until the water activity (aw) is below about 0.8.

17. The process according to claim 2, wherein the dried particles include a microorganism having a viability of at least 1.0×10E4 per gram as defined by the most probable number (MPN).

18. The process according to claim 2, further comprising packaging the dried particles in a package that is one or both of air-tight and moisture-tight.

19. The process according to claim 2, wherein steps (a) to (d) are performed in the presence of less than about 0.5% oxygen.

20. The process according to claim 2, wherein steps (a) to (d) are performed in the presence of a gas selected from nitrogen gas, a noble gas, carbon dioxide gas, and an alkane gas.

21. The process according to claim 2, wherein drying the purified frozen particles is conducted under reduced pressure.

22. The process according to claim 1, wherein steps (a) to (c) are performed in the presence of a concentration of oxygen selected from less than about 0.25% oxygen, less than about 0.1% oxygen, less than about 0.05% (about 5 ppm) oxygen, less than about 0.02% oxygen, less than about 0.03% oxygen, and less than about 0.04% oxygen.

23. The process according to claim 2, wherein steps (d) and (e) are performed in the presence of a concentration of oxygen selected from less than about 0.5% oxygen, less than about 0.25% oxygen, less than about 0.1% oxygen, less than about 0.05% (about 5 ppm) oxygen, less than about 0.02% oxygen, less than about 0.03% oxygen, and less than about 0.04% oxygen.
